(19) United States
(12) Patent Application Publication
(10) Pub. No.: US 2009/0239926 A1
(43) Pub. Date: Sep. 24, 2009

(54) METHODS FOR THE TREATMENT OF PSORIASIS OR PSORIATIC ARTHRITIS USING CYCLOPROPYL-N-CARBOXAMIDE

(76) Inventors: Peter H. Schafer, Somerset, NJ (US); George W. Muller, Bridgewater, NJ (US); Patricia E.W. Rohane, Florham Park, NJ (US)

Correspondence Address: JONES DAY, 222 E. 41ST. STREET, NEW YORK, NY, 10017, US

(21) Appl. No.: 12/410,011

(22) Filed: Mar. 24, 2009

Related U.S. Application Data
(60) Provisional application No. 61/070,514, filed on Mar. 24, 2008.

(51) Int. Cl. A61K 31/4035; A61P 17/06; A61K 39/395

(52) U.S. Cl. 514/416; 424/178.1

(57) ABSTRACT

Methods of treating, managing or preventing psoriasis or psoriatic arthritis are disclosed. Specific methods encompass the administration of cyclopropyl-N-{2-[(1S)-1-(3-ethoxy-4-methoxyphenyl)-2-(methylsulfonyl)ethyl]-3-oxoisoindoline-4-yl}carboxamide, alone or in combination with a second active agent. Pharmaceutical compositions and single unit dosage forms are also disclosed.

This application claims the benefit of U.S. provisional application No. 61/070,514, filed Mar. 24, 2008, the entireties of which are incorporated herein by reference.

### 1. FIELD

Provided herein are methods for treating, preventing and/or managing psoriasis by the administration of cyclopropyl-N-{2-[(1S)-1-(3-ethoxy-4-methoxyphenyl)-2-(methylsulfonyl)ethyl]-3-oxoisoindoline-4-yl}carboxamide, substantially free of its (R) enantiomer, alone or in combination with other therapeutics.

Provided also herein are methods of treating, preventing and/or managing psoriatic arthritis by the administration of cyclopropyl-N-{2-[(1S)-1-(3-ethoxy-4-methoxyphenyl)-2-(methylsulfonyl)ethyl]-3-oxoisoindoline-4-yl}carboxamide, substantially free of its (R) enantiomer, alone or in combination with other therapeutics. Provided also herein are pharmaceutical compositions and dosage forms comprising specific amounts of cyclopropyl-N-{2-[(1S)-1-(3-ethoxy-4-methoxyphenyl)-2-(methylsulfonyl)ethyl]-3-oxoisoindoline-4-yl}carboxamide suitable for use in methods of treating, preventing and/or managing psoriasis or psoriatic arthritis.

### 2. BACKGROUND

### 2.1 Psoriasis and Psoriatic Arthritis

Psoriasis is a chronic autoimmune inflammatory skin disorder characterized by epidermal hyperproliferation of keratinocytes and endothelial cells, and inflammatory cell accumulation (e.g., activated T cells). Griffiths C E, J. Eur. Acad. Dermatol. Venereol. 2003, 17 Suppl 2:1-5; Creamer J D, et al., Clin. Exp. Dermatol. 1995, 20(1):6-9. Also, recent evidence suggests the involvement of natural killer (NK) and NK T cells in the pathogenesis of psoriasis as these cells produce interferon-gamma (IFN-γ) which has been shown to play a role in psoriasis keratinocyte proliferation. Bos J D, et al., Br. J. Dermatol. 2005, 152(6):1098-107.

Clinically the main symptoms of psoriasis are gray or silvery flaky patches on the skin that are red and inflamed underneath. Central to the proposed pathogenic pathway are cytokines, chemokines and other inflammatory mediators produced by activated keratinocytes, dendritic cells, neutrophils, and NK T cells which are believed to induce both keratinocytes proliferation and lymphocyte migration. Creamer J D, et al., Clin. Exp. Dermatol. 1995, 20(1):6-9; Bos J D, et al., Br. J. Dermatol. 2005, 152(6):1098-107; Bowcock et al., Nat. Rev. Immunol. 2005, 5(9):699-71. Pro-inflammatory mediators shown to be elevated in the psoriasis skin lesions include, tumor necrosis factor-alpha (TNF-α), interleukin-6 (IL-6), IL-8, IL-12, IFN-γ, and inducible nitric oxide synthase (iNOS). LaDuca J R, et al., Dermatol. Clin. 2001, 19(4):617-35; Duan H, et al., J. Dermatol. Sci. 2001, 26(2):119-24; Gottlieb et al., J. Immunol. 2005, 175(4):2721-9. Furthermore, low expression levels of the anti-inflammatory cytokine IL-10 were observed in psoriasis lesions. Asadullah K, et al., Curr. Drug Targets Inflamm. Allergy. 2004, 3(2): 185-92.

Since the pathogenesis of psoriasis involves upregulation of at least TNF-α, IFN-γ, IL-6, IL-8 and IL-12 in addition to reductions in IL-10, PDE4 inhibitors may provide therapeutic benefits in the treatment of psoriasis.

Psoriatic arthritis is a chronic inflammatory arthritic condition affecting the skin, the joints, the insertion sites of tendons, ligaments, and fascia. Gladman, Current Opinion in Rheumatology, “Current concepts in psoriatic arthritis,” 2002, 14:361-366, and Ruddy et al., Rheumatology, vol. 2., chapter 71, page 1071, 6^{th} ed., 2001. Psoriatic arthritis is commonly associated with psoriasis. Id. Approximately 7% of patients with psoriasis develop psoriatic arthritis. The Merck Manual, 448 (17^{th} ed., 1999).

Psoriatic arthritis may appear in a variety of clinical patterns. There are five general patterns of psoriatic arthritis: arthritis of the distal interphalangeal joints, destructive arthritis, symmetric polyarthritis indistinguishable from rheumatoid arthritis, asymmetric oligoarthritis, and spondyloarthropathy. Ruddy et al., page 1073. Psoriasis appears to precede the onset of psoriatic arthritis in 60-80% of patients. Occasionally, arthritis and psoriasis appear simultaneously. Cutaneous eruptions may be preceded by the arthropathy.

Symptoms of psoriatic arthritis include extra bone formation, joint stiffness, dactylitis, enthesopathy, tendonitis, and spondylitis. Gladman, page 362. Most patients have the classic psoriasis pattern of skin lesions. Ruddy et al., page 1075. Scaly, erythematous plaques; guttate lesions, lakes of pus, and erythroderma are psoriatic skin lesions that may be seen in patients with psoriatic arthritis. Nail lesions, including pitting, Beau lines, leukonychia, onycholysis, oil spots, subungual hyperkeratosis, splinter hemorrhages, spotted lunulae, and cracking, are clinical features significantly associated with the development of psoriatic arthritis. Ruddy et al., page 1076. Ocular symptoms in psoriatic arthritis include conjunctivitis, iritis, episcleritis, keratoconjunctivitis sicca and aortic insufficiency.

Although the exact cause of psoriatic arthritis is unknown, genetic, environmental, immunologic, and vascular factors contribute to one's predisposition. Ruddy et al., pages 1071-72, and Gladman, page 363. The disease is more likely to occur in first-degree relatives who are affected than in the general population. Ruddy et al., page 1071. Population studies have shown that multiple human leukocyte antigens (HLA) are associated. British Society for Rheumatology, Rheumatology, 2001; 40:243, and Gladman, page 362. Much evidence suggests that a T-cell-mediated process drives the pathophysiology of psoriatic arthritis. Ruddy et al., pages 1071 and 1077, and Gladman, page 363. Activated T cells may contribute to the enhanced production of cytokines found in synovial fluid. Th1 cytokines (e.g., tumor necrosis factor-alpha (TNF-alpha), interleukin (IL)-1-beta and IL-10) are more prevalent in psoriatic arthritis than in rheumatoid arthritis, suggesting that the two diseases may result from a different mechanism. Ruddy et al., page 1071. Monocytes also play a role in psoriatic arthritis and are responsible for the production of matrix metalloproteinases, which may mediate the destructive changes in the joints of patients with psoriatic arthritis. Gladman, page 364.

Internationally, the incidence of psoriatic arthritis is 1-40%. Psoriatic arthritis usually develops in the fourth to sixth decades of life, but it can occur at almost any age. Men and women are affected equally, but a male predominance occurs in the spondylitic form, while a female predominance occurs in the rheumatoid form. Ruddy et al., page 1077.

There is a significant need for safe and effective methods of treating, preventing and managing psoriasis and psoriatic arthritis, particularly for patients that are refractory to conventional treatments. In addition, there is a need to treat such disease while reducing or avoiding the toxicity and/or side effects associated with conventional therapies.

### 3. SUMMARY

In one aspect, provided herein are methods for treating methods of treating, preventing and/or managing psoriasis or psoriatic arthritis in humans in need thereof. The methods comprise administering to a patient in need of such treatment, prevention or management a therapeutically or prophylactically effective amount of cyclopropyl-N-{2-[(1S)-1-(3-ethoxy-4-methoxyphenyl)-2-(methylsulfonyl)ethyl]-3-oxoisoindoline-4-yl}carboxamide (“Compound A”), or a pharmaceutically acceptable prodrug, metabolite, polymorph, salt, solvate (e.g., hydrate) or clathrate thereof, substantially free of its (R) enantiomer.

In some embodiments, the methods further comprise the administration of a therapeutically or prophylactically effective amount of at least a second active agent, including but not limited to, an anti-inflammatory agent, an immnunosuppressant, mycophenolate mofetil, a biologic agent, or a Cox-2 inhibitor.

In another embodiment, cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable prodrug, metabolite, polymorph, salt, solvate (e.g., hydrate) or clathrate thereof is administered orally in a dosage form such as a tablet and a capsule.

In further embodiments, cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable prodrug, metabolite, polymorph, salt, solvate (e.g., hydrate) or clathrate thereof is administered topically in a dosage form such as ointments, creams, gels, pastes, dusting powders, lotions, sprays, liniments, poultices, aerosols, solutions, emulsions and suspensions.

Particular embodiments herein provide pharmaceutical compositions for treating, preventing and/or managing psoriasis or psoriatic arthritis comprising cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable prodrug, metabolite, polymorph, salt, solvate (e.g., hydrate) or clathrate thereof.

Provided herein are single unit dosage forms for treating, preventing and/or managing psoriasis or psoriatic arthritis comprising cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable prodrug, metabolite, polymorph, salt, solvate (e.g., hydrate) or clathrate thereof.

The preferred methods and compositions utilize the salt or solvate, most preferably the free base of cyclopropyl-N-{2-[(1S)-1-(3-ethoxy-4-methoxyphenyl)-2-(methylsulfonyl)ethyl]-3-oxoisoindoline-4-yl}carboxamide.

### 4. BRIEF DESCRIPTION OF FIGURES

FIG. 1 illustrates the mean group arthritogenic scoring values of both hind paws (left and right average value) in mAb/LPS-induced mice arthritis model.

FIG. 2 illustrates mean group values of hind paw thickness in mAB/LPS-induced mice arthritogenic model.

FIG. 3 shows the effectiveness of Compound A in reducing the clinical severity of arthritis in type II collagen-induced mice arthritis model.

FIG. 4 shows the effectiveness of Compound A in reducing the histological severity of arthritis in type II collagen-induced mice arthritis model.

FIG. 5 shows the comparison between Compound A and rolipram of effect on spontaneous behaviors in non-arthritic mice model.

FIG. 6 shows in vitro inhibitions of cytokine production and T cell proliferation in untreated collagen-immunized mice by Compound A.

FIG. 7 shows inhibition of TNF-α production by synoviocytes from rheumatoid arthritis patients by Compound A.

FIG. 8 shows inhibition of LPS-stimulated monocyte TNF-α production by Compound A in a dose-dependent manner.

FIG. 9 shows inhibition of LPS-stimulated human PBMC TNF-α production by Compound A.

FIG. 10 shows inhibition of LPS-stimulated human whole blood TNF-α production by Compound A.

FIG. 11 shows inhibition of PDE4 enzymatic activity by Compound A.

FIG. 12 shows the elevation of cAMP by Compound A in PGE2-stimulated human PBMC.

FIG. 13 shows the inhibition of CD4⁺ IL-5 production by Compound A.

FIG. 14 shows the inhibition of fMLF-induced LTB4 production by compound A.

FIG. 15 shows the inhibition of fMLF-induced neutrophils CD18 and CD11b expression by Compound A.

FIG. 16 shows epidermal thickness in normal human skin xenotransplanted and psoriatic patient NK cells injected mice treated with Compound A or cyclosporine.

FIG. 17 shows keratinocyte proliferation index in normal human skin xenotransplanted and psoriatic patient NK cells injected mice treated with Compound A or cyclosporine.

FIG. 18 shows TNF-α expression in normal human skin graphs from psoriatic patient NK cells injected mice treated with Compound A or cyclosporine.

FIG. 19 shows HLA-DR expression in normal human skin graphs from psoriatic patient NK cells injected mice treated with Compound A or cyclosporine.

FIG. 20 shows ICAM-1 expression in normal human skin graphs from psoriatic patient NK cells injected mice treated with Compound A or cyclosporine.

### 5. DETAILED DESCRIPTION

### 5.1 Definitions

As used herein, the term “Compound A” refers to cyclopropyl-N-{2-[(1S)-1-(3-ethoxy-4-methoxyphenyl)-2-(methylsulfonyl)ethyl]-3-oxoisoindoline-4-yl}carboxamide.

As used herein and unless otherwise indicated, the term “pharmaceutically acceptable salt” includes, but is not limited to, salts prepared from pharmaceutically acceptable non-toxic acids or bases including inorganic acids and bases and organic acids and bases. Suitable pharmaceutically acceptable base addition salts for the compound of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts.

As used herein and unless otherwise indicated, the term “hydrate” means a compound of the present invention or a salt thereof, that further includes a stoichiometric or non-stoichiometeric amount of water bound by non-covalent intermolecular forces.

As used herein and unless otherwise indicated, the term “solvate” means a solvate formed from the association of one or more solvent molecules to a compound of the present invention. The term “solvate” includes hydrates (e.g., mono-hydrate, dihydrate, trihydrate, tetrahydrate and the like).

As used herein and unless otherwise indicated, the term “polymorph” means solid crystalline forms of a compound of the present invention or complex thereof. Different polymorphs of the same compound can exhibit different physical, chemical and/or spectroscopic properties.

As used herein and unless otherwise specified, the term “prodrug” means a derivative of a compound that can hydrolyze, oxidize, or otherwise react under biological conditions (in vitro or in vivo) to provide the compound. Examples of prodrugs include, but are not limited to, derivatives and metabolites of cyclopropyl-N-{2-[(1S)-1-(3-ethoxy-4-methoxyphenyl)-2-(methylsulfonyl)ethyl]-3-oxoisoindoline-4-yl}carboxamide that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by 1 Burger's Medicinal Chemistry and Drug Discovery, 172-178, 949-982 (Manfred E. Wolff ed., 5th ed. 1995).

As used herein, and unless otherwise specified, the term “enantiomer,” “isomer” or “stereoisomer” encompasses all enantiomerically/stereomerically pure and enantiomerically/stereomerically enriched compounds of this invention.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure, when the compound contains greater than or equal to 80%, 90%, 95%, 98% or 99% of one stereoisomer, and 20%, 10%, 5%, 2%, 1% or less of the counter stereoisomer. “Substantially free of its (R) enantiomer” is encompassed by the term stereomerically pure or enantiomerically pure.

As used herein, term “adverse effect” includes, but is not limited to gastrointestinal, renal and hepatic toxicities, leukopenia, increases in bleeding times due to, e.g., thrombocytopenia, and prolongation of gestation, nausea, vomiting, somnolence, asthenia, dizziness, teratogenicity, extra-pyramidal symptoms, akathisia, cardiotoxicity including cardiovascular disturbances, inflammation, male sexual dysfunction, and elevated serum liver enzyme levels. The term “gastrointestinal toxicities” includes but is not limited to gastric and intestinal ulcerations and erosions. The term “renal toxicities” includes but is not limited to such conditions as papillary necrosis and chronic interstitial nephritis.

As used herein, the term “patient” refers to a mammal, particularly a human. In some embodiments, the patient is a female. In further embodiments, the patient is a male. In further embodiments, the patient is a child.

As used herein, and unless otherwise specified, the terms “treat,” “treating” and “treatment” contemplate an action that occurs while a patient is suffering from the specified disease or disorder, which reduces the severity or symptoms of the disease or disorder, or retards or slows the progression or symptoms of the disease or disorder.

As used herein, unless otherwise specified, the terms “prevent,” “preventing” and “prevention” contemplate an action that occurs before a patient begins to suffer from the specified disease or disorder, which inhibits or reduces the severity or symptoms of the disease or disorder.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

### 5.2 Methods of Treatments and Prevention

Provided herein are methods of treating, managing and/or preventing psoriasis or psoriatic arthritis, which comprise administering to a patient in need of such treatment, management or prevention a therapeutically or prophylactically effective amount of cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable prodrug, metabolite, polymorph, salt, solvate or clathrate thereof. Preferably the salt or solvate, most preferably the free base of cyclopropyl-N-{2-[(1S)-1-(3-ethoxy-4-methoxyphenyl)-2-(methylsulfonyl)ethyl]-3-oxoisoindoline-4-yl}carboxamide, is used in the methods.

In certain embodiments, the methods comprise administering cyclopropyl-N-{2-[(1S)-1-(3-ethoxy-4-methoxyphenyl)-2-(methylsulfonyl)ethyl]-3-oxoisoindoline-4-yl}carboxamide, substantially free of its (R) enantiomer, or a pharmaceutically acceptable prodrug, metabolite, polymorph, salt, solvate or clathrate of thereof, after the onset of symptoms of psoriasis or psoriatic arthritis.

In certain embodiments, the methods also encompass inhibiting or averting symptoms of psoriasis or psoriatic arthritis as well as addressing the disease itself, prior to the onset of symptoms by administering cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable prodrug, metabolite, polymorph, salt, solvate or clathrate thereof. Patients having history of psoriasis or psoriatic arthritis are preferred candidates for preventive regimens. Methods comprise administering cyclopropyl-N-{2-[(1S)-1-(3-ethoxy-4-methoxyphenyl)-2-(methlsulfonl)ethyl]-3-oxoisoindoline-4-yl}carboxamide, or a pharmaceutically acceptable prodrug, metabolite, polymorph, salt, solvate or clathrate thereof, to a patient (e.g., a human) suffering or likely to suffer, from psoriasis or psoriatic arthritis.

The magnitude of a prophylactic or therapeutic dose of cyclopropyl-N-{2-[(1S)-1-(3-ethoxy-4-methoxyphenyl)-2-(methylsulfonyl)ethyl]-3-oxoisoindoline-4-yl}carboxamide in the acute or chronic management of psoriasis or psoriatic arthritis, will vary with the nature and severity of the disease or condition, and the route by which the compound is administered. The dose, and perhaps the dose frequency, will also vary according to the age, body weight, and response of the individual patient. Suitable dosing regimens can be readily selected by those skilled in the art with due consideration of such factors. In general, the recommended daily dose range for the conditions described herein lie within the range of from about 1 mg to about 1,000 mg per day, given as a single once-a-day dose or as divided doses throughout a day. More specifically, the daily dose is administered twice, three times or four times daily in equally divided doses. Specifically, a daily dose range may be from about 5 mg to about 500 mg per day, more specifically, between about 10 mg and about 200 mg per day. Specifically, the daily dose may be administered in 5 mg, 10 mg, 15 mg, 20 mg, 25 mg, 50 mg, 100 mg or 200 mg dosage forms. In managing the patient, the therapy may be initiated at a lower dose, perhaps about 1 mg to about 25 mg, and increased if necessary up to about 200 mg to about 1,000 mg per day as either a single dose or divided doses, depending on the patient's global response. In further embodiments, the daily dose of cyclopropyl-N-{2-[(1S)-1-(3-ethoxy-4-methoxyphenyl)-2-(methylsulfonyl)ethyl]-3-oxoisoindoline-4-yl}carboxamide is from about 0.01 mg to about 100 mg per kg of a body weight of a patient. In some embodiments, the daily dose of the compound is about 1 mg/kg, 5 mg/kg, 10 mg/kg or 25 mg/kg.

### 5.2.1 Combination Therapy with a Second Active Agent or Therapy

In particular methods encompassed by this embodiment, cyclopropyl-N-{2-[(1S)-1-(3-ethoxy-4-methoxyphenyl)-2-(methylsulfonyl)ethyl]-3-oxoisoindoline-4-yl}carboxamide is administered in combination with another drug (“second active agent”) for treating, managing and/or preventing psoriatic arthritis or psoriasis.

Cyclopropyl-N-{2-[(1S)-1-(3-ethoxy-4-methoxyphenyl)-2-(methylsulfonyl)ethyl]-3-oxoisoindoline-4-yl}carboxamide can be combined with one or more second active agents in methods. In certain embodiments, the methods encompass synergistic combinations for the treatment, prevention and/or management of psoriasis or psoriatic arthritis. Cyclopropyl-N-{2-[(1S)-1-(3-ethoxy-4-methoxyphenyl)-2-(methylsulfonyl)ethyl]-3-oxoisoindoline-4-yl}carboxamide can also be used to alleviate adverse or unnamed effects associated with some second active agent. Conversely, some second active agents can be used to alleviate adverse or unnamed effects associated with cyclopropyl-N-{2-[(1S)-1-(3-ethoxy-4-methoxyphenyl)-2-(methylsulfonyl)ethyl]-3-oxoisoindoline-4-yl}carboxamide.

One or more second active agents can be used in the methods together with cyclopropyl-N-{2-[(1S)-1-(3-ethoxy-4-methoxyphenyl)-2-(methylsulfonyl)ethyl]-3-oxoisoindoline-4-yl}carboxamide. The second active agents include, but are not limited to, anti-inflammatories such as nonsteroidal anti-inflammatory drugs (NSAIDs), immnunosuppressants, mycophenolate mofetil, biologic agents, and Cox-2 inhibitors.

The second active agents can be administered before, after or simultaneously with cyclopropyl-N-{2-[(1S)-1-(3-ethoxy-4-methoxyphenyl)-2-(methylsulfonyl)ethyl]-3-oxoisoindoline-4-yl}carboxamide.

In some embodiments of interest, the second active agents may include, but are not limited to, anti-inflammatories such as NSAIDs including, but not limited to, diclofenac (e.g., ARTHROTEC®), diflunisal (e.g., DOLOBID®), etodolac (e.g., LODINE®) fenoprofen (e.g., NALFON®), ibuprofen (e.g., ADVIL, CHILDREN'S ADVIL/MOTRIN, MEDIPREN, MOTRIN, NUPRIN or PEDIACARE FEVER®), indomethacin (e.g., ARTHREXIN®), ketoprofen (e.g., ORUVAIL®), ketorolac (e.g., TORADOL®), fosfomycin tromethamine (e.g., MONURAL®), meclofenamate (e.g., Meclomen®), nabumetone (e.g., RELAFEN®), naproxen (e.g., ANAPROX®, ANAPROX® DS, EC-NAPROSYN®, NAPRELAN® or NAPROSYN®), oxaprozin (e.g., DAYPRO®), piroxicam (e.g., FELDENE®), sulindac (e.g., CLINORIL®), and tolmetin (e.g., TOLECTIN® DS or TOLECTIN®).

In other embodiments of interest, the second active agents may include, but are not limited to, disease-modifying antirheumatic drugs (DMARDs) or immnunosuppressants such as, but not limited to, methotrexate (Rheumatrex®), sulfasalazine (Azulfidine), and cyclosporine (Sandimmune® or Neroal®).

In other embodiments of interest, the second active agents may include, but are not limited to, mycophenolate mofetil (CellCept®). It is an immunosuppressive agent widely used in organ transplantation and gaining favor in treating autoimmune and inflammatory skin disorders.

In further embodiments of interest, the second active agents may include, but are not limited to, biologic agents such as, but not limited to, etanercept (Enbrel®), infliximab (Remicade®) and adalimumab (Humira®).

In further embodiments of interest, the second active agents may include, but are not limited to, Cox-2 inhibitors such as, but not limited to, celecoxib (Celebrex®), valdecoxib (Bextra®) and meloxicam (Mobic®).

Administration of cyclopropyl-N-{2-[(1S)-1-(3-ethoxy-4-methoxyphenyl)-2-(methylsulfonyl)ethyl]-3-oxoisoindoline-4-yl}carboxamide and a second active agent to a patient can occur simultaneously or sequentially by the same or different routes of administration. The suitability of a particular route of administration employed for a particular second active agent will depend on the second active agent itself (e.g., whether it can be administered orally or topically without decomposition prior to entering the blood stream) and the subject being treated. A particular route of administration of cyclopropyl-N-{2-[(1S)-1-(3-ethoxy-4-methoxyphenyl)-2-(methylsulfonyl)ethyl]-3-oxoisoindoline-4-yl}carboxamide is oral administration in dosage forms of a tablet or a capsule. Particular routes of administration for the second active agents or ingredients are known to those of ordinary skill in the art. See, e.g., The Merck Manual, 448 (17^{th} ed., 1999).

The amount of second active agent administered can be determined based on the specific agent used, the subject being treated, the severity and stage of disease and the amount(s) of cyclopropyl-N-{2-[(1S)-1-(3-ethoxy-4-methoxyphenyl)-2-(methylsulfonyl)ethyl]-3-oxoisoindoline-4-yl}carboxamide and any optional additional second active agents concurrently administered to the patient. Those of ordinary skill in the art can determine the specific amounts according to conventional procedures known in the art. In the beginning, one can start from the amount of the second active agent that is conventionally used in the therapies and adjust the amount according to the factors described above. See, e.g., Physician's Desk Reference (59^{th} Ed., 2005).

In certain embodiments, the second active agent is administered orally, topically, intravenously or subcutaneously and once to four times daily in an amount of from about 1 to about 1,000 mg, from about 5 to about 500 mg, from about 10 to about 350 mg or from about 50 to about 200 mg. The specific amount of the second active agent will depend on the specific agent used, the age of the subject being treated, the severity and stage of disease and the amount(s) of cyclopropyl-N-{2-[(1S)-1-(3-ethoxy-4-methoxyphenyl)-2-(methylsulfonyl)ethyl]-3-oxoisoindoline-4-yl}carboxamide and any optional additional second active agents concurrently administered to the patient. In one embodiment, cyclopropyl-N-{2-[(1S)-1-(3-ethoxy-4-methoxyphenyl)-2-(methylsulfonyl)ethyl]-3-oxoisoindoline-4-yl}carboxamide can be administered in an amount of from about 1 mg to about 1,000 mg, preferably from about 5 mg to about 500 mg, and more preferably from about 10 mg and about 200 mg orally and daily alone or in combination with a second active agent disclosed herein (see, e.g., section 5.2.1), prior to, during or after the use of conventional therapy. In another embodiment, the daily dose of cyclopropyl-N-{2-[(1S)-1-(3-ethoxy-4-methoxyphenyl)-2-(methylsulfonyl)ethyl]-3-oxoisoindoline-4-yl}carboxamide is from about 0.01 mg to about 100 mg per kg of a body weight of a patient.

### 5.3 Cyclopropyl-N-{2-[1(1S)-1-(3-Ethoxy-4-Methoxyphenyl)-2-(methylsulfonyl)Ethyl]1-3-Oxoisoindoline-4-}carboxamide

In certain embodiments, the methods of treating, managing or preventing psoriasis or psoriatic arthritis comprise administering to a patient in need of such treatment, management or prevention a therapeutically or prophylactically effective amount of cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable prodrug, metabolite, polymorph, salt, solvate or clathrate thereof. Without being limited by theory, cyclopropyl-N-{2-[(1S)-1-(3-ethoxy-4-methoxyphenyl)-2-(methylsulfonyl)ethyl]-3-oxoisoindolin-4-yl}carboxamide is believed to be (S) enantiomer, which has the following structure:

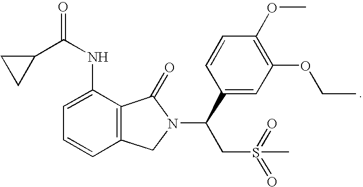

Cyclopropyl-N-{2-[(1S)-1-(3-ethoxy-4-methoxyphenyl)-2-(methylsulfonyl)ethyl]-3-oxoisoindoline-4-yl}carboxamide can be prepared according to methods disclosed in U.S. Pat. No. 6,667,316, titled “Pharmaceutically active isoindoline derivatives,” and U.S. Provisional application No. 60/851,152 filed on Oct. 11, 2006, titled “PROCESS FOR THE PREPARATION OF 2-(1-PHENYLETHYL)ISOINDOLIN-1-ONE COMPOUNDS,” the entireties of which are incorporated herein by reference.

Generally, cyclopropyl-N-{2-[(1S)-1-(3-ethoxy-4-methoxyphenyl)-2-(methylsulfonyl)ethyl]-3-oxoisoindoline-4-yl}carboxamide can be readily prepared using the methods described in U.S. Pat. No. 6,667,316 and U.S. Provisional application No. 60/851,152, which are incorporated herein by reference. The (S) enantiomer can be isolated from the racemic compound by techniques known in the art. Examples include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N.Y., 1962); and Wilen, S. H., Tables of Resolving Agents and Optical Resolutions p. 268 (E. L. Eliel, Ed., Univ. of Notre Dame Press, Notre Dame, Ind., 1972).

In a specific method, cyclopropyl-N-{2-[1-(3-ethoxy-4-methoxyphenyl)-2-(methylsulfonyl)ethyl]-3-oxoisoindoline-4-yl}carboxamide can be prepared, for example, by reacting Compound (6) with cyclopropylcarbonyl chloride in the presence of N,N-diisopropylethylamine. The acylation reaction can occur, for example, at a reaction temperature between 20° C. and 25° C. for about 4 and about 6 hours in ethyl acetate. The mole ratio of Compound (6) to cyclopropylcarbonyl chloride to N,N-diisopropylethylamine is about 1:1.05:1.2.

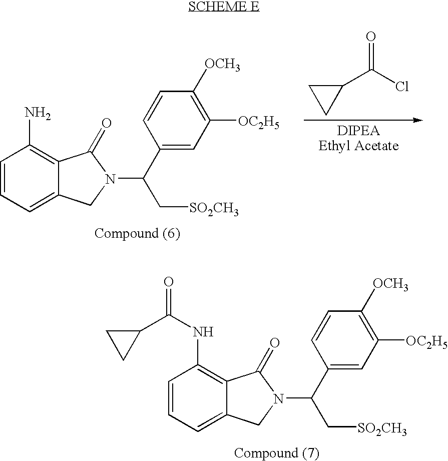

An enantiomerically pure Compound (6) can be used for the preparation of an enantiomerically pure compound (7). Alternatively, a racemic mixture of Compound (7) can be prepared and then resolved into the enantiomers by conventional resolution techniques such as biological resolution and chemical resolution.

### 5.4 Pharmaceutical Compositions and Dosage Forms

Pharmaceutical compositions can be used in the preparation of individual, single unit dosage forms. Pharmaceutical compositions and dosage forms can comprise cyclopropyl-N-{2-[(1S)-1-(3-ethoxy-4-methoxyphenyl)-2-(methylsulfonyl)ethyl]-3-oxoisoindoline-4-yl}carboxamide or a pharmaceutically acceptable salt or solvate thereof and a second active agent. Examples of the optional second active agents are disclosed herein (see, e.g., section 5.2.1). Pharmaceutical compositions and dosage forms can further comprise one or more carriers, excipients or diluents.

Single unit dosage forms are suitable for oral, mucosal (e.g., nasal, sublingual, vaginal, cystic, rectal, preputial, ocular, buccal or aural), parenteral (e.g., subcutaneous, intravenous, bolus injection, intramuscular or intraarterial), topical (e.g., eye drops or other ophthalmic preparations), transdermal or transcutaneous administration to a patient. Non-limiting examples of dosage forms include tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions or a water-in-oil liquid emulsions), solutions and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms will vary from one another will be readily apparent to those skilled in the art. See, e.g., Remington's Pharmaceutical Sciences, 20th ed., Mack Publishing, Easton Pa. (2,000).

Typical pharmaceutical compositions and dosage forms comprise one or more excipients. Suitable excipients are well known to those skilled in the art of pharmacy and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients can be accelerated by some excipients such as lactose or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions can comprise excipients that are well known in the art and are listed, for example, in the U.S. Pharmacopeia (USP) 25-NF20 (2002). In general, lactose-free compositions comprise active ingredients, a binder/filler and a lubricant in pharmaceutically compatible and pharmaceutically acceptable amounts. Particular lactose-free dosage forms comprise active ingredients, microcrystalline cellulose, pre-gelatinized starch and magnesium stearate.

In certain embodiments, provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, New York, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Non-limiting examples of suitable packaging include hermetically sealed foils, plastics, unit dose containers (e.g., vials), blister packs and strip packs.

Provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers or salt buffers. Like the amounts and types of excipients, the amounts and specific types of active ingredients in a dosage form may differ depending on factors such as, but not limited to, the route by which it is to be administered to patients. However, typical dosage forms comprise cyclopropyl-N-{2-[(1S)-1-(3-ethoxy-4-methoxyphenyl)-2-(methylsulfonyl)ethyl]-3-oxoisoindoline-4-yl}carboxamide or a pharmaceutically acceptable salt or solvate thereof in an amount of from about 1 to about 1,000 mg. Typical dosage forms comprise cyclopropyl-N-{2-[(1S)-1-(3-ethoxy-4-methoxyphenyl)-2-(methylsulfonyl)ethyl]-3-oxoisoindoline-4-yl}carboxamide or a pharmaceutically acceptable salt or solvate thereof in an amount of about 1, 2, 5, 7.5, 10, 12.5, 15, 17.5, 20, 25, 50, 100, 150 or 200 mg. In a particular embodiment, a dosage form comprises cyclopropyl-N-{2-[(1S)-1-(3-ethoxy-4-methoxyphenyl)-2-(methylsulfonyl)ethyl]-3-oxoisoindoline-4-yl}carboxamide in an amount of about 1, 5, 10, 25, 50, 100 or 200 mg.

### 5.4.1 Oral Dosage Forms

Provided herein are pharmaceutical compositions that are suitable for oral administration can be presented as discrete dosage forms, such as, but not limited to, tablets (e.g., chewable tablets), caplets, capsules and liquids (e.g., flavored syrups). Such dosage forms contain predetermined amounts of active ingredients and can be prepared by methods of pharmacy well known to those skilled in the art. See generally, Remington 's Pharmaceutical Sciences, 20th ed., Mack Publishing, Easton Pa. (2,000).

Typical oral dosage forms are prepared by combining the active ingredients in an intimate admixture with at least one excipient according to conventional pharmaceutical compounding techniques. Excipients can take a wide variety of forms depending on the form of preparation desired for administration. Non-limiting examples of excipients suitable for use in oral liquid or aerosol dosage forms include water, glycols, oils, alcohols, flavoring agents, preservatives and coloring agents. Non-limiting examples of excipients suitable for use in solid oral dosage forms (e.g., powders, tablets, capsules and caplets) include starches, sugars, micro-crystalline cellulose, diluents, granulating agents, lubricants, binders and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers or both and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Non-limiting examples of excipients that can be used in oral dosage forms include binders, fillers, disintegrants and lubricants. Non-limiting examples of binders suitable for use in pharmaceutical compositions and dosage forms include corn starch, potato starch or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose and mixtures thereof.

Non-limiting examples of suitable forms of microcrystalline cellulose include, but are not limited to, the materials sold as AVICEL® (microcrystalline cellulose) PH-101, AVICEL® (microcrystalline cellulose) PH-103, AVICEL RC-581® (crystalline cellulose and carboxymethylcellulose sodium), AVICEL® (microcrystalline cellulose) PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581® (crystalline cellulose and carboxymethylcellulose sodium). Suitable anhydrous or low moisture excipients or additives include AVICEL-PH-103™® (microcrystalline cellulose) PH-103 and Starch 1500® LM (pregelatinized starch).

Non-limiting examples of fillers suitable for use in the pharmaceutical compositions and dosage forms disclosed herein include talc, calcium carbonate (e.g., granules or powder), microcrystalline cellulose, powdered cellulose, dextrates, kaolin, mannitol, silicic acid, sorbitol, starch, pre-gelatinized starch and mixtures thereof. The binder or filler in pharmaceutical compositions is typically present in from about 50 to about 99 weight percent of the pharmaceutical composition or dosage form.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation and is readily discernible to those of ordinary skill in the art. Typical pharmaceutical compositions comprise from about 0.5 to about 15 weight percent of disintegrant, preferably from about 1 to about 5 weight percent of disintegrant.

Non-limiting examples of disintegrants that can be used in pharmaceutical compositions and dosage forms include agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums and mixtures thereof.

Non-limiting examples of lubricants that can be used in pharmaceutical compositions and dosage forms include calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g, peanut oil, cottonseed oil, sunflower oil, sesame oil, olive oil, corn oil and soybean oil), zinc stearate, ethyl oleate, ethyl laureate, agar and mixtures thereof. Additional lubricants include, for example, a syloid silica gel (AEROSIL200®(silica), manufactured by W. R. Grace Co. of Baltimore, Md.), a coagulated aerosol of synthetic silica (marketed by Degussa Co. of Plano, Tex.), CAB-O-SIL® (fumed silica) (a pyrogenic silicon dioxide product sold by Cabot Co. of Boston, Mass.) and mixtures thereof. If used at all, lubricants are typically used in an amount of less than about 1 weight percent of the pharmaceutical compositions or dosage forms into which they are incorporated.

A particular solid oral dosage form comprises cyclopropyl-N-{2-[(1S)-1-(3-ethoxy-4-methoxyphenyl)-2-(methylsulfonyl)ethyl]-3-oxoisoindoline-4-yl}carboxamide, anhydrous lactose, microcrystalline cellulose, polyvinylpyrrolidone, stearic acid, colloidal anhydrous silica and gelatin.

### 5.4.2 Delayed Release Dosage Forms

In certain embodiments, active ingredients can be administered by controlled release means or by delivery devices that are well known to those of ordinary skill in the art. Non-limiting examples of controlled release means or delivery devices include those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556 and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres or a combination thereof to provide the desired release profile in varying proportions. Suitable controlled-release formulations known to those of ordinary skill in the art, including those described herein, can be readily selected for use with the active ingredients. In certain embodiments, provided herein are single unit dosage forms suitable for oral administration such as, but not limited to, tablets, capsules, gelcaps and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency and increased patient compliance. In addition, controlled-release formulations can be used to affect the time of onset of action or other characteristics, such as blood levels of the drug and can thus affect the occurrence of side (e.g., adverse) effects.

Most controlled-release formulations are designed to initially release an amount of drug (active ingredient) that promptly produces the desired therapeutic effect and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water or other physiological conditions or compounds.

### 5.4.3 Parenteral Dosage Forms

Parenteral dosage forms can be administered to patients by various routes including, but not limited to, subcutaneous, intravenous (including bolus injection), intramuscular and intraarterial. Because their administration typically bypasses patients' natural defenses against contaminants, parenteral dosage forms are preferably sterile or capable of being sterilized prior to administration to a patient. Non-limiting examples of parenteral dosage forms include solutions ready for injection, dry products ready to be dissolved or suspended in a pharmaceutically acceptable vehicle for injection, suspensions ready for injection and emulsions.

Suitable vehicles that can be used to provide parenteral dosage forms are well known to those skilled in the art. Non-limiting examples of suitable vehicles include Water for Injection USP; aqueous vehicles such as, but not limited to, Sodium Chloride Injection, Ringer's Injection, Dextrose Injection, Dextrose and Sodium Chloride Injection and Lactated Ringer's Injection; water-miscible vehicles such as, but not limited to, ethyl alcohol, polyethylene glycol and polypropylene glycol; and non-aqueous vehicles such as, but not limited to, corn oil, cottonseed oil, peanut oil, sesame oil, ethyl oleate, isopropyl myristate and benzyl benzoate.

Compounds that increase the solubility of one or more of the active ingredients disclosed herein can also be incorporated into the parenteral dosage forms. For example, cyclodextrin and its derivatives can be used to increase the solubility of cyclopropyl-N-{2-[(1S)-1-(3-ethoxy-4-methoxyphenyl)-2-(methylsulfonyl)ethyl]-3-oxoisoindoline-4-yl}carboxamide and its derivatives.

### 5.4.4 Topical and Mucosal Dosage Forms

Drugs can be applied locally to the skin and its adnexa or to a variety of mucous membranes. The routes that can be used include nasal, sublingual, vaginal, cystic, rectal, preputial, ocular, buccal or aural. Many dosage forms have been developed to deliver active principles to the site of application to produce local effects. Non-limiting examples of topical and mucosal dosage forms include sprays, inhalers, aerosols, ointments, creams, gels, pastes, dusting powders, lotions, liniments, poultices, solutions, emulsions, suspensions, eye drops or other ophthalmic preparations or other forms known to one of skill in the art. See, e.g., Remington's Pharmaceutical Sciences, 20^{th} ed., Mack Publishing, Easton Pa. (2,000); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms are well known to those skilled in the pharmaceutical arts and depend on the particular tissue to which a given pharmaceutical composition or dosage form will be applied. Non-limiting examples of typical excipients include water, acetone, ethanol, ethylene glycol, propylene glycol, butane-1,3-diol, isopropyl myristate, isopropyl palmitate, mineral oil and mixtures thereof to form solutions, emulsions or gels, which are non-toxic and pharmaceutically acceptable.

Moisturizers such as occlusives, humectants, emollients and protein rejuvenators can also be added to pharmaceutical compositions and dosage forms if desired. Examples of such additional ingredients are well known in the art. See, e.g., Remington 's Pharmaceutical Sciences, 20^{th} ed., Mack Publishing, Easton Pa. (2,000).

Occlusives are substances that physically block water loss in the stratum corneum. Non-limiting examples of occlusives include petrolatum, lanolin, mineral oil, silicones such as dimethicone, zinc oxide and combinations thereof. Preferably, the occlusives are petrolatum and lanolin, more preferably petrolatum in a minimum concentration of 5%.

Humectants are substances that attract water when applied to the skin and theoretically improve hydration of the stratum corneum. However, the water that is drawn to the skin is water from other cells, not atmospheric water. With this type of moisturizer, evaporation from the skin can continue and actually can make the dryness worse. Non-limiting examples of humectants include glycerin, sorbitol, urea, alpha hydroxy acids, sugars and combinations thereof. Preferably, the humectants are alpha hydroxy acids, such as glycolic acid, lactic acid, malic acid, citric acid and tartaric acid.

Emollients are substances that smooth skin by filling spaces between skin flakes with droplets of oil, and are not usually occlusive unless applied heavily. When combined with an emulsifier, they may help hold oil and water in the stratum comeum. Vitamin E is a common additive, which appears to have no effect, except as an emollient. Likewise, other vitamins, for example, A and D, are also added, but their effect is questionable. Non-limiting examples of emollients include mineral oil, lanolin, fatty acids, cholesterol, squalene, structural lipids and combinations thereof.

Protein rejuvenators are substances that rejuvenate the skin by replenishing essential proteins. Non-limiting examples of protein rejuvenators include collagen, keratin, elastin and combinations thereof.

The pH of a pharmaceutical composition or dosage form may also be adjusted to improve delivery of one or more active ingredients. Similarly, the polarity of a solvent carrier, its ionic strength or tonicity can be adjusted to improve delivery. For example, absorption through the skin can also be enhanced by occlusive dressings, inunction or the use of dimethyl sulfoxide as a carrier. Compounds such as metal stearates (e.g., calcium stearate, zinc stearate, magnesium stearate, sodium stearate, lithium stearate, potassium stearate, etc.) can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant and as a delivery-enhancing or penetration-enhancing agent. Different salts, hydrates or solvates of the active ingredients can be used to further adjust the properties of the resulting composition.

### 6. EXAMPLES

Some embodiments are illustrated by the following non-limiting examples. The examples should not be construed as a limitation in the scope thereof. The scope of the invention is defined solely by the appended claims.

### 6.1 Example 1

### mAB/LPS-Induced Mice Arthritis Model

The anti-arthritic activity of Compound A was assessed in the mAB/LPS-induced experimental arthritis in male BALB/c mice.

mAb/LPS-induced Mice Arthritis Model: Experimental arthritis in the tested mice was initially induced on Day 0 by a single intravenous injection (IV) into tail vein of monoclonal antibodies (mAb) cocktail at a dose level of 100 mg/kg, followed about 72 hours later by a single intraperitoneal (IP) injection of lipopolysaccharide (LPS) 2.5 mg/kg.

Treatment Regimen: Compound A was administered by oral gavage (PO), using a suitable stainless steel feeding needle. Enbrel (Reference Item) was administrated by intraperitoneal (IP) injections. Compound A was first administered on study Day 3 (one hour prior to the single LPS injection) and thereafter on Days 4, 5, 6, 7, 8, 9, 10, 11, 12 and 13 (total of 11 successive treatment days) at 1, 5 and 25 mg/kg once daily. Compound A treated groups comprised n=8 BALB/c male mice per dosing group. In addition, two equally sized groups were treated with either Enbrel (5 mg/kg/day, Reference Item), or a solution of 0.5% Na CMC/0.25% Tween 80 (PO, 5 ml/kg, Vehicle Control).

No obvious treatment-related adverse reactions were observed among all treated animals throughout the entire 14-day observation period, excluding the typical reactions to LPS injection, characterized by piloerection, decrease in the spontaneous motor activity and slight diarrhea.

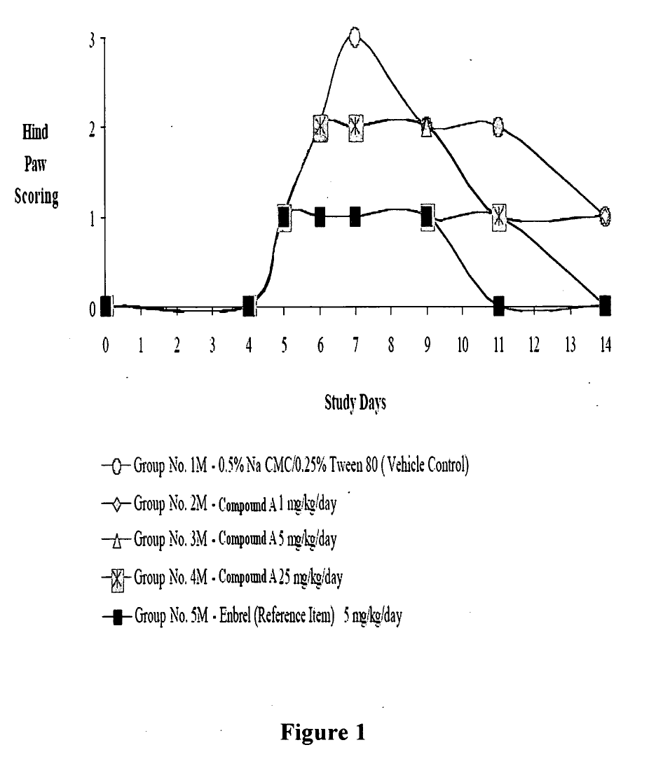

Arthritis Reactions: Both hind paws (left and right) of each animal were examined for signs of arthritogenic responses prior to arthritis induction (Day 0) and thereafter on study days 4, 5, 6, 7, 9, 11 & 14 as selected by the Sponsor. Arthritis reactions were scored and recorded according to a 0-4 scale in ascending order of severity (based on Morwell MD Biosciences Inc. brochure) as shown in Table 1. The results are shown in FIG. 1 and Table 2. The arthritogenic scoring values in animals subjected to 25 mg/kg/day of Compound A were found to be statistically lower (p<0.05) on Days 9 and 14.

TABLE 1
Standard of Arthritis Reaction Scoring

| Arthritis Score | Grade |
|---|---|
| No reaction, normal | 0 |
| Mild, but definite redness and swelling of the ankle or apparent | 1 |
| redness and swelling limited to individual digits. | |
| regardless of the number of affected digits | |
| Moderate redness and swelling of ankle | 2 |
| Severe redness and swelling of the entire paw including digits | 3 |
| Maximally inflamed limb with involvement of multiple joints | 4 |

TABLE 2
Arthritis Scoring on mAb/LPS-induced Murine Arthritis Model

| Group | | Dose Level | | | | | | | | Day | Day |
|---|---|---|---|---|---|---|---|---|---|---|---|
| No. | Treatment | (mg/kg/day) | | Day 0 | Day 4 | Day 5 | Day 6 | Day 7 | Day 9 | 11 | 14 |
| 1 | Vehicle | 0 | Mean ± | 0 | 0 | 1 | 2 | 3 | 2 | 2 | 1 |
| | Control | | SD | 0.0 | 0.0 | 0.6 | 0.5 | 0.3 | 0.4 | 0.6 | 0.7 |
| 5 | Enbrel | 5 | Mean ± | 0 | 0 | 1 | 1 | 1**↓ | 1***↓ | 0***↓ | 0**↓ |
| | | | SD | 0.0 | 0.0 | 0.7 | 0.9 | 1.1 | 0.7 | 0.4 | 0.4 |
| 2 | Compound A | 1 | Mean ± | 0 | 0 | 1 | 2 | 2 | 2 | 1 | 1 |
| | | | SD | 0.0 | 0.0 | 0.7 | 1.0 | 0.6 | 0.5 | 0.8 | 0.4 |
| 3 | | 5 | Mean ± | 0 | 0 | 1 | 2 | 2 | 2 | 1 | 1 |
| | | | SD | 0.0 | 0.0 | 0.6 | 0.5 | 0.4 | 0.6 | 0.6 | 0.5 |
| 4 | | 25 | Mean ± | 0 | 0 | 1 | 2 | 2 | 1*↓ | 1 | 0*↓ |
| | | | SD | 0.0 | 0.0 | 0.9 | 1.1 | 1.3 | 1.0 | 0.8 | 0.5 |

0 - No reaction, normal
1 - Mild, but definite redness and swelling of the ankle or apparent redness and swelling limited to individual digits, regardless of the number of affected digits
2 - Moderate redness and swelling of ankle
3 - Severe redness and swelling of the entire paw including digits
4 - Maximally inflamed limb with involvement of multiple joints
*↓P < 0.05 vs. Vehicle Control (Kruskal-Wallis Nonparametric Test)
**↓P < 0.01 vs. Vehicle Control (Kruskal-Wallis Nonparametric Test)
***↓P < 0.001 vs. Vehicle Control (Kruskal-Wallis Nonparametric Test)

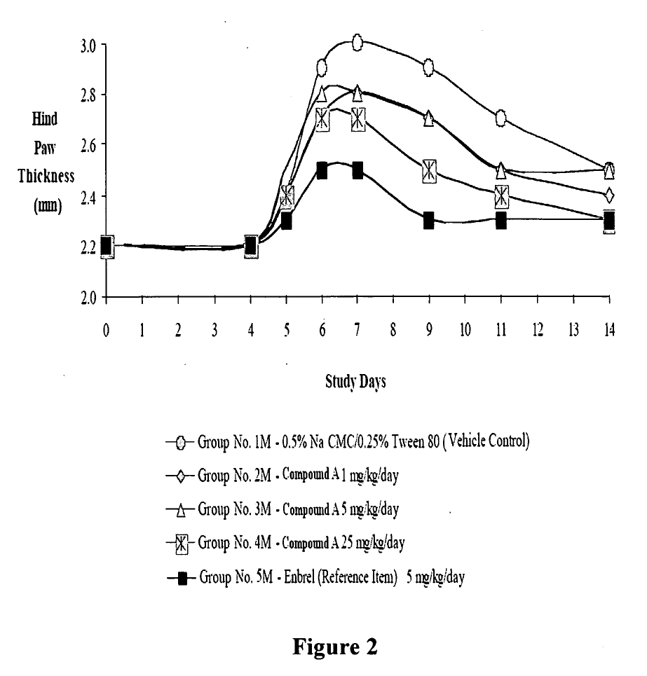

Measurements of Experimental Arthritis: Hind paw thickness was determined on eight days using a Mitutoyo Electronic Digital Caliper (on Days 0, 4, 5, 6, 7, 9, 11 and 14) and presented as mean group values of the average for both left and right hind paws. The results are shown in FIG. 2 and Table 3. Data indicated highly significant decrease (P<0.0l vs. Vehicle Control) in animals subjected to repeated administrations of 25 mg/kg/day of the Compound A on Days 9, 11 and 14. In animals subjected to repeated administrations of 1 and 5 mg/kg/day of Compound A, statistically significant values (p<0.05) were revealed on Day 11.

TABLE 3
Hind Paws Thickness (mm) on Study Days

| Group | | Dose Level | | | | | | | | Day | Day |
|---|---|---|---|---|---|---|---|---|---|---|---|
| No. | Treatment | (mg/kg/day) | | Day 0 | Day 4 | Day 5 | Day 6 | Day 7 | Day 9 | 11 | 14 |
| 1 | Vehicle | 0 | Mean ± | 2.2 | 2.2 | 2.4 | 2.9 | 3.0 | 2.9 | 2.7 | 2.5 |
| | Control | | SD | 0.06 | 0.07 | 0.12 | 0.25 | 0.30 | 0.28 | 0.16 | 0.14 |
| 5 | Enbrel | 5 | Mean ± | 2.2 | 2.2 | 2.3 | 2.5*↓ | 2.5**↓ | 2.3**↓ | 2.3**↓ | 2.3**↓ |
| | | | SD | 0.04 | 0.04 | 0.16 | 0.29 | 0.30 | 0.15 | 0.11 | 0.10 |
| 2 | Compound A | 1 | Mean ± | 2.2 | 2.2 | 2.4 | 2.7 | 2.8 | 2.7 | 2.5*↓ | 2.4 |
| | | | SD | 0.05 | 0.05 | 0.15 | 0.29 | 0.23 | 0.21 | 0.17 | 0.08 |
| 3 | | 5 | Mean ± | 2.2 | 2.2 | 2.5 | 2.8 | 2.8 | 2.7 | 2.5*↓ | 2.5 |
| | | | SD | 0.02 | 0.02 | 0.11 | 0.17 | 0.15 | 0.16 | 0.13 | 0.11 |
| 4 | | 25 | Mean ± | 2.2 | 2.2 | 2.4 | 2.7 | 2.7 | 2.5**↓ | 2.4**↓ | 2.3**↓ |
| | | | SD | 0.04 | 0.05 | 0.23 | 0.36 | 0.39 | 0.27 | 0.19 | 0.11 |

*↓P < 0.05 vs. Vehicle Control (1-Way ANOVA Dunnett Multiple Comparison Test)
**↓P < 0.01 vs. Vehicle Control (1-Way ANOVA Dunnett Multiple Comparison Test)

Mean group percentage changes in hind paw thickness vs. arthritis induction initiation (Day 0) was found to be highly significantly lower (P<0.01) in animals subjected to repeated administrations of 25 mg/kg of Compound A on Days 9, 11 and 14. The data is shown Table 4.

TABLE 4
Percentage Change (%) in Hind Paw Thickness on Study Days vs. Study
Commencement (Day 0)

| Group | | Dose Level | | | | | | | Day | Day |
|---|---|---|---|---|---|---|---|---|---|---|
| No. | Treatment | (mg/kg/day) | | Day 4 | Day 5 | Day 6 | Day 7 | Day 9 | 11 | 14 |
| 1 | Vehicle | 0 | Mean ± | 0 | 11 | 30 | 35 | 32 | 21 | 14 |
| | Control | | SD | 0.8 | 5.0 | 13.2 | 14.8 | 13.5 | 8.5 | 7.6 |
| 5 | Enbrel | 5 | Mean ± | 0 | 6 | 13 | 12**↓ | 7**↓ | 4**↓ | 4**↓ |
| | | | SD | 0.0 | 6.7 | 13.7 | 14.0 | 7.0 | 4.8 | 4.5 |
| 2 | Compound A | 1 | Mean ± | 1 | 8 | 24 | 27 | 23 | 13 | 7 |
| | | | SD | 1.1 | 5.4 | 13.2 | 11.1 | 10.2 | 8.4 | 4.7 |
| 3 | | 5 | Mean ± | 1 | 12 | 30 | 30 | 21 | 15 | 13 |
| | | | SD | 1.1 | 5.3 | 7.9 | 7.3 | 7.5 | 5.8 | 4.9 |
| 4 | | 25 | Mean ± | 0 | 8 | 21 | 21 | 13**↓ | 8**↓ | 5**↓ |
| | | | SD | 0.8 | 9.4 | 16.1 | 17.5 | 12.4 | 8.5 | 4.8 |

**↓P < 0.01 vs. Vehicle Control (1-Way ANOVA Dunnett Multiple Comparison Test)

### 6.2 Example 2

### Type II Collagen-Induced Mice Arthritis Model

This study was performed to test Compound A for therapeutic effects against collagen-induced arthritis in male mice.

Collagen-Induced Arthritis Animal Model: Type II collagen purified from the cartilage of a young calf was dissolved at 4 mg/ml in 0.1M acetic acid and emulsified with an equal volume of complete Freund's adjuvant (CFA). DBA/1 mice (8-12 weeks of age) were then immunized at two sites at the base of the tail on Day 1 with 100 μl of the emulsion.

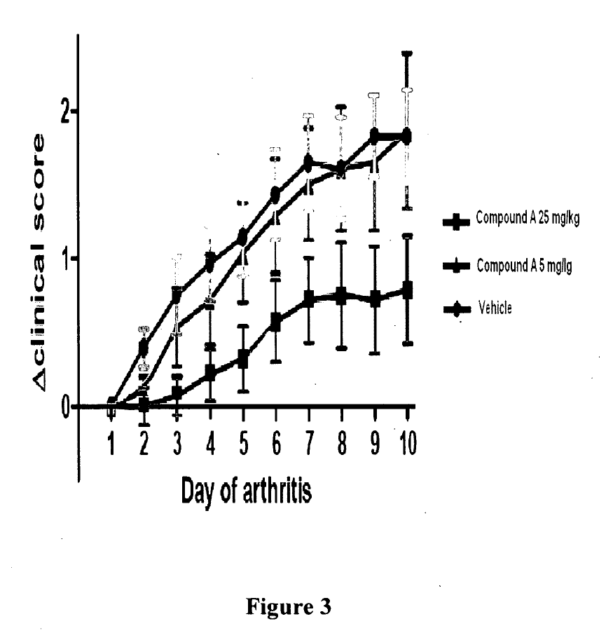

Treatment regime and measurement of clinical score: 14 animals per test group were treated orally once a day for 10 days (Days 1-10) with vehicle (0.5% carboxymethyl-cellulose/0.25% Tween 80), or Compound A suspended in vehicle at 5 mg/kg or 25mg/kg. The experiment was terminated on Day 10. Mice were monitored daily for signs of arthritis and scored using an established clinical scoring system, where: 0=normal, 1=slight swelling and/or erythema, and 2=pronounced edematous swelling. Each limb was graded, giving a maximum score of 8 per mouse. In addition, paw-swelling was measured using calipers. Results show that Compound A was effective in reducing the clinical severity of arthritis at 25 mg/kg ( FIG. 3).

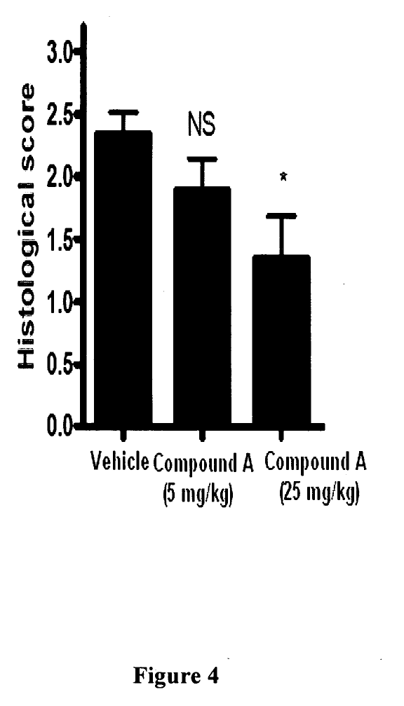

Histology: At the end of the experiment, paws from treated mice were fixed in formal saline, decalcified and embedded in wax, sectioned and stained with hematoxylin and eosin. Histopathological assessment of arthritis was carried out in a blinded fashion on hematoxylin- and eosin-stained sections using a scoring system as follows: 0, normal; 1, minimal synovitis without cartilage/bone erosion; 2, synovitis with some marginal erosion but joint architecture maintained; 3, severe synovitis and erosion with loss of normal joint architecture. Results show that Compound A reduced the histological severity of arthritis at 25 mg/kg (P<0.05, FIG. 4).

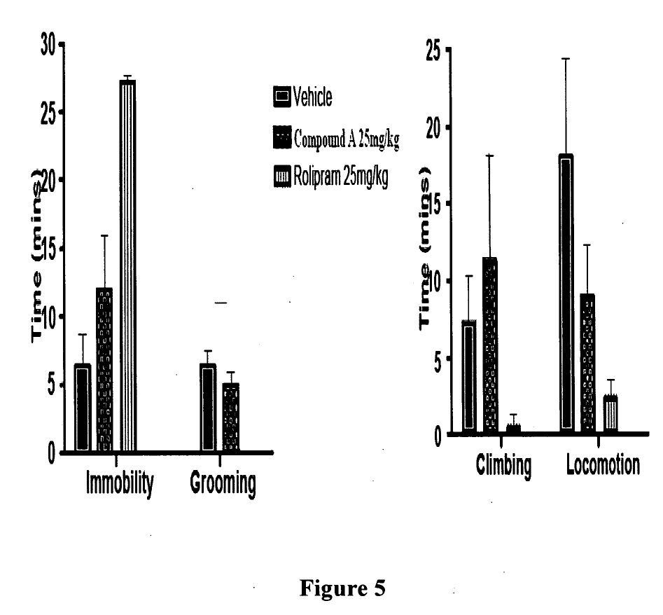

Behavioral studies: The effect of Compound A (25 mg/kg/day) on spontaneous behavior was assessed using the LABORAS (Laboratory Animal Behavior Observation Registration and Analysis System), which is an automated system that detects vibrations evoked by movement of a single rodent in a cage. Pattern recognition software then recognizes and quantifies behaviors, including grooming, activity, climbing, immobility, and feeding. Compound A had little or no effect on grooming, time spent immobile or climbing, whilst causing only a modest reduction in locomotion (FIG. 5).

Ex Vivo Procedure: Mice were bled and lymph nodes were excised. Lymph node cells (LNC) were stimulated in vitro with antigen (type II collagen) or mitogen (anti-CD3 mAb) in the presence of Compound A.

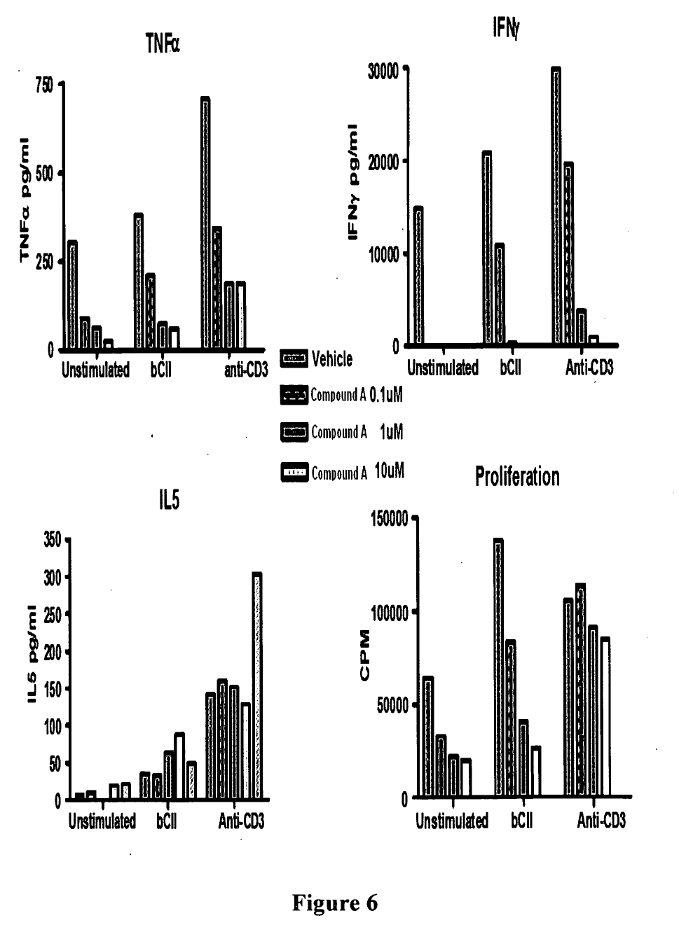

Profound effects on both proliferation and T cell cytokine production were observed. The results show that Compound A inhibited T cell proliferation, IFN-γ and TNF-α production in a dose-dependent fashion. In contrast, production of the Th2 cytokine, IL-5, was unaffected (FIG. 6).

Conclusion: Compound A is effective in reducing arthritis severity at the clinical and histological levels. Importantly, Compound A did not have any major effects on spontaneous behavior, suggesting that this compound would be tolerated much better than traditional PDE4 inhibitors, such as rolipram.

### 6.3 Example 3

### TNF-Alpha Inhibition

Human Rheumatoid Synovial Membrane Cell: Rheumatoid synovial membrane tissue samples were processed to dissociate the cells from the matrix by digesting the tissue with Collagenase A and DNAse. The cells were then plated into a 96-well flat-bottom plate at 1×10⁶/well in RPMI (10% FCS) and treated with Compound A and controls (in triplicate). The cells were cultured for 48 hours at 37° C. in 5% CO₂ before supernatants were harvested and analyzed by ELISA.

Compound A was solubilized under sterile conditions in filter-sterilized dimethyl sulphoxide (DMSO). The vehicle control contained the same concentration of DMSO used as the diluent in the highest concentration of drug used. A combined treatment of anti-TNF-α and IL-1RA, both at 10 μg/ml, was used as a positive control.

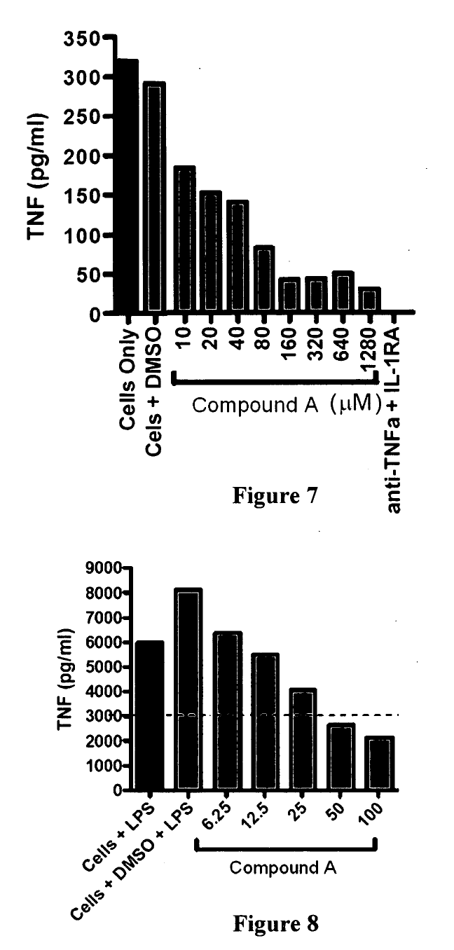

Compound A effectively inhibited TNF-α production in a dose-dependent manner (FIG. 7). IC₅₀ of Compound A was 100 nM.

LPS-induced TNF-α production in Monocytes: Monocytes separated from human peripheral blood mononuclear cells (PBMCs) were plated into a 96-well flat bottom plate at 1×10⁵/well in RPMI (5% heat inactivated fetal calf serum (FCS) and then treated with increasing concentrations of Compound A and control (in triplicate). Following a 30 minute pre-incubation period, the monocytes were stimulated with LPS (10 ng/ml) and cultured for 24 hours at 37° C. in 5% CO₂. The supernatants are then harvested and analyzed by ELISA.

The results show that Compound A inhibited LPS-stimulated monocytes TNF-α production in a dose-dependent manner. Compound A has a monocyte TNF-α IC₅₀ value of 40 nM (FIG. 8).

LPS-induced TNF-α production in Human PBMC: Human peripheral blood mononuclear cells (PBMC) (2×10⁵ cells) were plated in 96-well flat-bottom Costar tissue culture plates (Corning, N.Y., USA) in triplicate. Compound A was dissolved in DMSO (Sigma) and further dilutions were done in culture medium immediately before use. The final DMSO concentration in all samples was 0.25%. Various concentration of Compound A was added to cells one hour before stimulation. Cells were stimulated with LPS (Sigma, St. Louis, Mo., USA) at 100 ng/ml, in the absence or presence of Compound A. Cells were incubated for 18-20 hours at 37 in 5% CO₂ and supernatants were then collected, diluted with culture medium and assayed for TNF-α levels by ELISA (Endogen, Boston, Mass., USA) (Muller, G. W., et al., J Med Chem, 1996. 39(17): 3238-40).

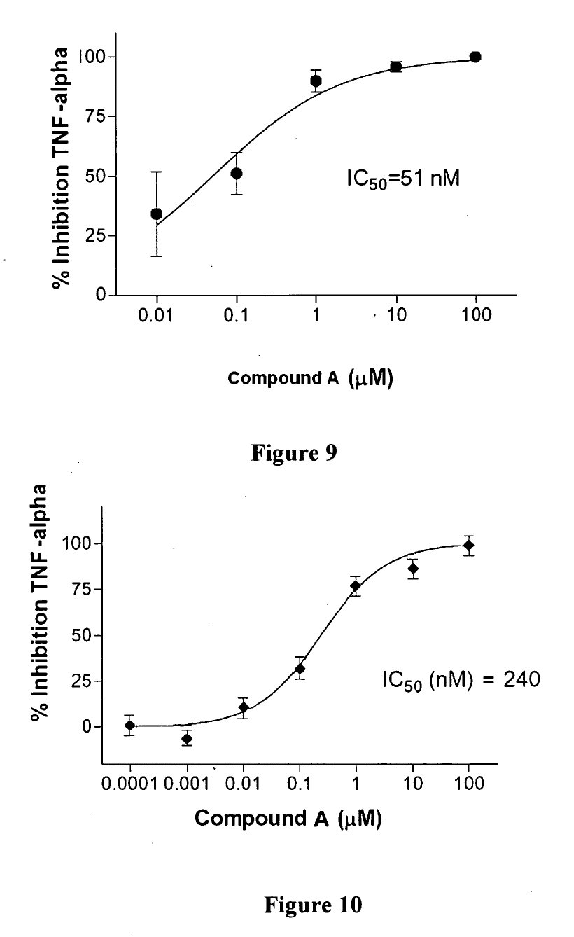

Results indicate that Compound A has a PBMC TNF-α IC₅₀ of 51 nM (24 ng/ml) (FIG. 9 and Table 5).

LPS-induced TNF-α production in Human Whole Blood: The ability of Compound A to inhibit LPS-induced TNF-α production by human whole blood was measured as described above for the LPS-induced TNF-α assay in human PBMC, except that freshly drawn whole blood was used instead of PBMC.

Compound A has a whole blood TNF-α IC₅₀ of 240 nM (110 ng/ml) (FIG. 10 and Table 5).

Mouse LPS-induced serum TNF-α production: Compound A was administered to female BALB/c mice orally by gavage two hours prior to LPS challenge. Blood was drawn 1.5 hours after LPS challenge, and serum TNF-α was measured as described above.

Compound A inhibited mouse LPS-induced serum TNF-α levels by 83% (n=2) at 1 mg/kg p.o., and 3% (n=2) at 0.1 mg/kg p.o. Based on these data, an ED₅₀ for this model would be between 0.1 and 1 mg/kg (Table 5).

### 6.4 Example 4

### PDE4 Inhibition

PDE4 enzyme was purified from U937 human monocytic cells by gel filtration chromatography (Muller et al. 1998, Bioorg. & Med. Chem. Lett. 1998. 8 (19):2669-74). Phosphodiesterase reactions were carried out in 50 MM Tris HCl pH 7.5, 5 mM MgCl₂, 1 μM cAMP, 10 nM [³H]-cAMP for 30 min at 30, terminated by boiling, treated with 1 mg/ml snake venom, and separated using AG-lXS ion exchange resin (BioRad).

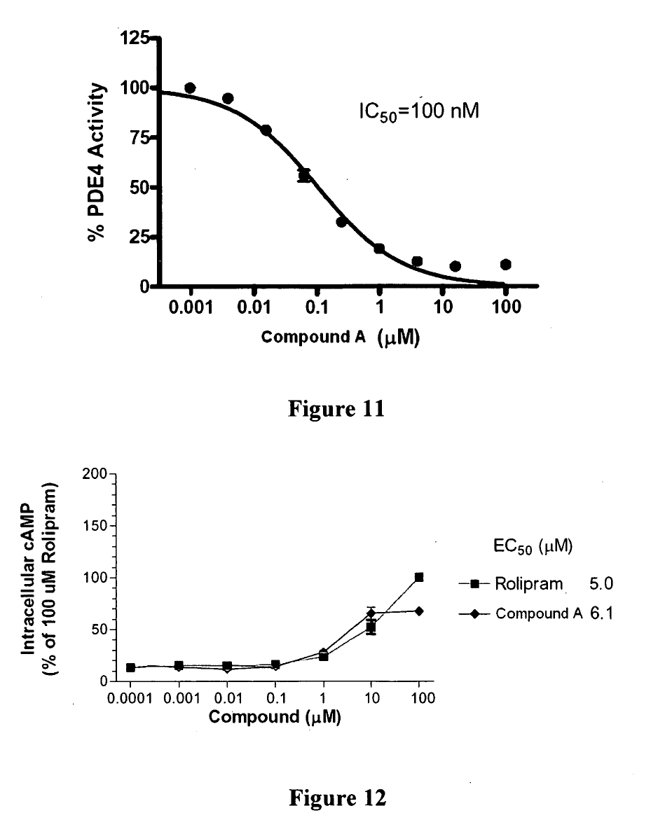

The results indicate that Compound A has a PDE4 IC₅₀ of 100 nM (50 ng/ml) (FIG. 11 and Table 5).

### 6.5 Example 5

### Specificity for PDE4 Inhibition

The specificity of Compound A for PDE4 was assessed by testing at a single concentration (10 μM) against bovine PDE1, human PDE2, PDE3 and PDE5 from human platelets, and PDE6 from bovine retinal rod outer segments. (Hidaka, H. and T. Asano, Biochim Biophys Acta, 1976. 429(2): 485-97; Nicholson, C. D., R. A. Challiss, and M. Shahid, Trends Pharmacol Sci, 1991. 12(1): 19-27; Baehr, W., M. J. Devlin, and M. L. Applebury, J Biol Chem, 1979. 254(22): 11699-77; and Gillespie, P. G. and J. A. Beavo, Mol Pharmacol, 1989. 36(5): 773-81). At 10 μM, Compound A inhibited PDE1 by 30%, PDE2 by −14%, PDE3 by 9%, PDE4 by 95%, PDE5 by −7%, and PDE6 by 17% (Table 5).

### 6.6 Example 6

### PGE2-Induced Camp Elevation

Prostaglandin E2 (PGE2) binds to prostanoid receptors on monocytes, T cells and other leukocytes and consequently elevates intracellular cAMP levels, resulting in inhibition of cellular responses. The combination of PGE2 and a PDE4 inhibitor synergistically elevates cAMP levels in these cell types, and the elevation of cAMP in PBMC caused by PDE4 inhibitors in the presence of PGE2 is proportional to the inhibitory activity of that PDE4 inhibitor.

Human PBMCs were isolated as described above and plated in 96-well plates at 1×10⁶ cells per well in RPMI-1640. The cells were pre-treated with Compound A in a final concentration of 2% DMSO in duplicate at 37° C. in a humidified incubator at 5% CO₂ for one hour. The cells were then stimulated with PGE2 (10 μM) (Sigma) for one hour. The cells were lysed with HCl, 0.1N final concentration to inhibit phosphodiesterase activity and the plates were frozen at −20° C. The cAMP produced was measured using cAMP (low pH) Immunoassay kit (R&D Systems).

Results indicate that Compound A has a PBMC cAMP EC₅₀ of 6.1 μM (2.9 μg/ml) (FIG. 12 and Table 5).

### 6.7 Example7

### IL-5 Production by CD4+ T Cells

CD4⁺ T cells were purified from human leukocytes obtained from the Blood Center of New Jersey (East Orange, N.J.) by negative selection (Schafer, P. H., et al., J Immunol, 1999. 162(12): 7110-9). CD4⁺ T cells were stimulated with CD3 antibody OKT3 (purified from OKT3 hybridoma supernatant) and CD28 antibody CD28.2 (BD Pharmingen) (Hatzelmann, A. and C. Schudt, J Pharmacol Exp Ther, 2001. 297(1): 267-79). IL-5 was measured by ELISA (R&D Systems).

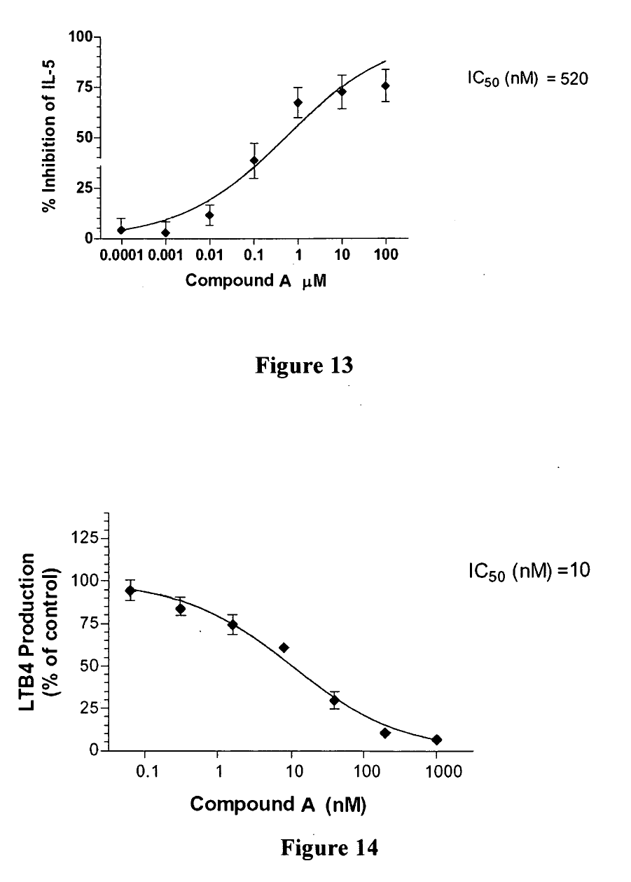

Results indicate that Compound A has an IL-5 IC₅₀ of 520 nM (250 ng/ml) (FIG. 13 and Table 5).

### 6.8 Example 8

### FMLF-Induced Neutrophil LTB4 Production

Formyl-Met-Leu-Phe (fMLP, Sigma) is a bacterially derived peptide that activates neutrophils to rapidly degranulate, migrate, and adhere to endothelial cells. Among the contents of the neutrophil granule is leukotriene B4 (LTB4), a product of arachidonic acid metabolism and itself a neutrophil stimulant. Compound A was tested for the ability to block fMLF-induced neutrophil LTB4 production.

Human neutrophils were isolated from human leukocyte units by dextran sedimentation as described in Coligan, J. E., et al., Current Protocols in Immunology, ed. R. Coico. Vol. 2. 2002: 2-3. The neutrophils were resuspended in phosphate-buffered saline without calcium or magnesium (BioWhittaker) containing 10 mM HEPES pH 7.2 and plated in 96-well tissue culture plates at a concentration of 1.7×10⁶ cells/well. Cells were treated with 50 μM thimerosal (Sigma)/1 mM CaCl₂/1 mM MgCl₂ for 15 minutes at 37° C. 5% CO₂, then treated with Compound A in a final DMSO concentration of 0.01% in duplicate for 10 minutes. Neutrophils were stimulated with 1 μM fMLP for 30 minutes, then lysed by the addition of methanol (20% final concentration) and frozen in a dry ice/isopropanol bath for 10 minutes. Lysates were stored at −70° C. until the LTB4 content was measured by competitive LTB4 ELISA (R&D systems).

Results indicate that Compound A has a LTB4 IC₅₀ of 10 nM (4.7 ng/ml) (FIG. 14 and Table 5).

### 6.9 Example 9

### FMLF-Induced Neutrophil CD18/CD11B Expression

CD18/CD11b (Mac-1) expression on neutrophils was measured with the following modifications. (Derian, C. K., et al., J Immunol, 1995. 154(1): 308-17). Neutrophils were isolated as described above, then resuspended in complete medium at 1×10⁶ cells/ml, pretreated with Compound A at 10, 1, 0.1, 0.01, and 0 μM in duplicate at a final DMSO concentration of 0.1% for 10 minutes at 37° C. 5% CO₂. Cells were then stimulated with 30 nM fMLF for 30 minutes and then chilled to 4° C. Cells were treated with rabbit IgG (Jackson ImmunoResearch Labs, West Grove, Pa., USA) (10 μg/1×10⁶ cells) to block Fc receptors, stained with CD18-FITC and CD11b-PE (Becton Dickinson), and analyzed by flow cytometry on a FACSCalibur. CD18/CD11b expression (mean fluorescence) in the absence of stimulation was subtracted from all samples to obtain inhibition curves and calculate IC₅₀.

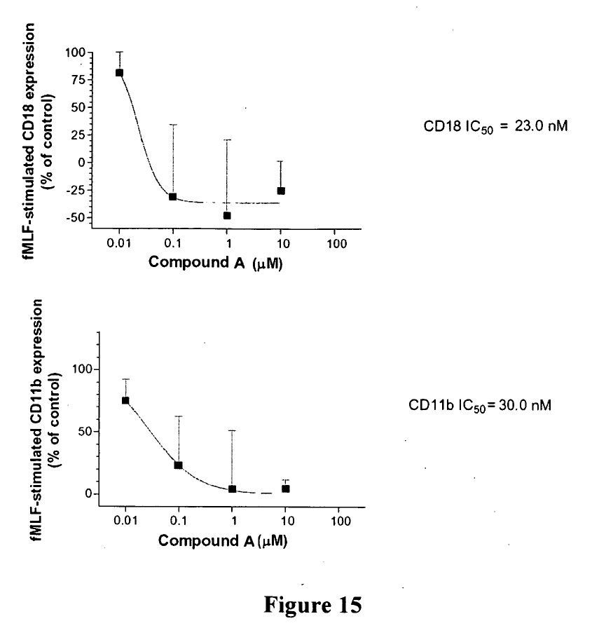

Results show that Compound A has an CD18 IC₅₀ of 23 nM (11 ng/ml) and a CD11b IC₅₀ of 30 nM (14 ng/ml) (FIG. 15 and Table 5).

TABLE 5
Summary of enzymatic, cellular, and in vivo data of Compound A

| | Compound A | |
|---|---|---|
| | IC₅₀ (nM) | IC₅₀ (ng/ml) |
| PDE4 IC₅₀ (from U937 cells) | 100 | 50 |
| PGE2-induced PBMC cAMP EC50 | 6,100 | 2,900 |
| LPS-induced PBMC TNF-α IC₅₀ | 51 | 24 |
| Human Whole Blood LPS-induced TNF-α IC₅₀ | 240 | 110 |
| fMLF-induced Neutrophil LTB4 IC₅₀ | 10 | 4.7 |
| fMLF-induced Neutrophil CD18 | 23 | 11 |
| expression IC₅₀ | | |
| fMLF-induced Neutrophil CD11b | 30 | 14 |
| expression IC₅₀ | | |
| CD4+ T cell IL-5 IC₅₀ | 520 | 250 |

| | |
|---|---|
| PDE1 (% inhibition at 10 μM) | 30% |
| PDE2 (% inhibition at 10 μM) | −14% |
| PDE3 (% inhibition at 10 μM) | 9% |
| PDE4 (% inhibition at 10 μM) | 95% |
| PDE5 (% inhibition at 10 μM) | −7% |
| PDE6 (% inhibition at 10 μM) | 17% |
| Mouse LPS-induced serum TNF-α | 0.1-1 |
| inhibition (ED₅₀, mg/kg, p.o.) | |

### 6.10 Example 10

### Treatments of Psoriasis

The purpose of this study was to compare the effectiveness of Compound A to cyclosporine, an approved treatment for severe plaque-type psoriasis, in a human NK cell-driven model of psoriasis that utilized human skin xenotransplanted onto beige-severe combined immunodeficiency (SCID) mice. Nickoloff B J, et al., Am. J. Pathol. 1995, 146(3):580-8; Wrone-Smith T, et al., J. Clin. Invest. 1996, 98(8):1878-87.

Six psoriatic patients were included in this study, mean age 42 years, ranged from 29 to 58 years. All patients had classic plaque psoriasis. None of the patients were previously treated. Normal skin from seven normal volunteers was also obtained for grafting.

Healthy human skin pieces having a width of 0.4 mm and surface area of 3×3 cm were provided from residual skin of routine plastic surgery procedures from the Plastic Surgery Department of the Rambam Medical Center, Isreal. In addition, blood samples from psoriatic patients were taken at a volume of 25 mL.

Twenty-one (21) beige-severe combined immunodeficient mice (SCID) (weight about 20 g) were included in this study. Normal human skin was transplanted onto the beige-SCID mice as previously described by Nickoloff et al., 1995; Wrone-Smith, et al., 1996. A sample of each donor was transplanted onto four mice so that the three treatment groups (n=7 mice/group) were homogenous.

PBMC from the psoriatic patient blood were isolated and cultured in the presence of IL-2 (100 U/mL of media) for 14 days to activate the NK cells, as previously described by Gilhar et al., J. Invest. Dermatol. 2002, 119(2):384-91. Four weeks following the engraftment, each mouse was injected with 1×10⁷ psoriatic patients activated allogeneic NK cells (1×10⁷ cells injected/mouse, n=21). Two weeks following the injections, the mice were divided and treated, twice a day for 14 days. All compounds were dosed at 5 mg/kg/day, divided into b.i.d. doses. To perform p.o. administration, mice were held firmly by gently gripping their fur over the neck with thumb and index fingers and restraining the tail with the little finger. A volume of 0.05 mL of a 1 mg/ml aqueous solution of Compound A was administrated twice a day (b.i.d.) with a syringe through a blunt-ended curved feeding tube, which was inserted into the esophagus. The vehicle (negative) control groups received 0.05 mL (b.i.d.) of a 0.5% carboxymethylcellulose and 0.25% Tween 80. Compound A and positive control (cyclosporine) groups received similar treatment administration. Two weeks after starting the treatments (4 weeks following the injections), the skins were harvested. Grafts were analyzed by histology and immunohistochemistry.

Determination of Epidermal Thickness: Skin graft histological assessment was performed by light microscopy both before and after transplantation and two blinded observers performed the evaluations. Epidermal thickness was determined with an ocular micrometer, at a minimum of 50 points along the epidermis selected to represent points of maximal and minimal thickness. Thickness of the suprapapillary plate was similarly measured at 50 points for each sample.

Immunohistochemical Staining: Monoclonal antibodies to human antigens used were as follows for immunohistochemistry on frozen sections: anti-HLA-DR (Becton Dickenson, San Jose, Calif.), and anti-CD54 (ICAM-1) (Biodesign, Saco, Me.). Purified murine IgG was used as a control for the above antibodies. Immunohistochemistry was performed on OCT embedded specimens with a biotin-avidin system (Vectostain, Vector Laboratories, Burlingame, Calif.).

Goat anti-human TNF-α (R&D Systems, Minneapolis, Minn.) was used on deparaffinized and peroxidase blocked slides. Sections were treated with citrate buffer, pH=6, in the microwave oven for 20 minutes. The sections were then cooled for 30 minutes at room temperature and blocked for non-specific binding as well as avidin-biotin. All washes were performed with phosphate-buffered saline-saponin. Anti-TNF-α was applied overnight at 4° C. Slides were then incubated with biotinylated rabbit anti goat-IgG (DAKO, Carpinteria, Calif.), followed by streptavidin horseradish peroxidase (HRP) (Jackson Immunoresearch, West Grove, Pa.). The color was developed with 3-amino-9-ethylcarbazole (AEC). The epidermal proliferation index was determined as a percentage of keratinocytes expressing Ki-67 as detected by the monoclonal anti-human Ki-67 antibody (Zymed Laboratories, San Francisco, Calif,, U.S.A.) using the above procedure, except that antigen retrieval was achieved with EDTA (pH=8) buffer.

Scoring of Immunohistochemical Staining: Diffuse staining was defined as positive and intense expression of more than 50 percent of the epidermis versus focal staining which was defined as less than 50 percent of the epidermis. Focal staining may represent positive expression of very small areas.

Statistical Analysis: Statistical analysis was carried out using the one-way ANOVA with a Bonferroni Multiple Comparison post-hoc test using Prism 4.00 (GraphPad Software; San Diego, Calif.).

Histological Evaluation of Normal Human Skin Grafts From Psoriatic NK cell Injected Beige-SCID Mice: The experiment was composed of 21 mice divided into three groups (n=7). All mice were injected with NK cells obtained from psoriatic patients, according to the protocol. Thereafter the mice were separated into to the vehicle, Compound A and cyclosporine treatment groups. Normal human skin xenotransplanted onto beige-SCID mice, treated with psoriatic NK cells and vehicle expressed psoriasiform features that included epidermal thickening (acanthosis), hyperkeratosis, parakeratosis, along with a dermal lymphocytic infiltrate, some areas with retention, others with lack of the granular layer. Additionally, elongation of rete ridges was observed in most psoriatic NK cells injected normal skin grafts and vascular dilatation associated with a perivascular lymphocytic infiltrate was noted in the papillary dermis. Therefore, normal skin grafts injected with psoriatic NK cells showed, in many cases, histological features similar to psoriasis but combined with some signs of dermatitis supporting the utility of the human skin xenotransplant/SCID mouse model of psoriasis.

Specifically, the histological evaluations of the normal human skin xenotransplanted to beige-SCID mice and injected with psoriatic patient NK cells demonstrated psoriasiform histological features in all seven specimens of the vehicle treatment group (Table 6 and 7). Histological evaluation of Compound A treatment group demonstrated that 1/7 mice displayed a partial recovery and 3/7 mice experienced a complete recovery from psoriasis features for an overall response rate of 57%. The cyclosporine treated group had a response rate of 42.9% ( 1/7 partial and 2/7 complete recovery), approximately 14 percentage points lower than Compound A (Table 6).

TABLE 6
Histological Evaluation of Human Skin Grafts Following Treatment

| Histological features | Vehicle | Compound A | Cyclosporine |
|---|---|---|---|
| Psoriasiform | 7/7 | 3/7 | 4/7 |
| Complete Recovery | 0/7 | 3/7 | 2/7 |
| Partial Recovery | 0/7 | 1/7 | 1/7 |

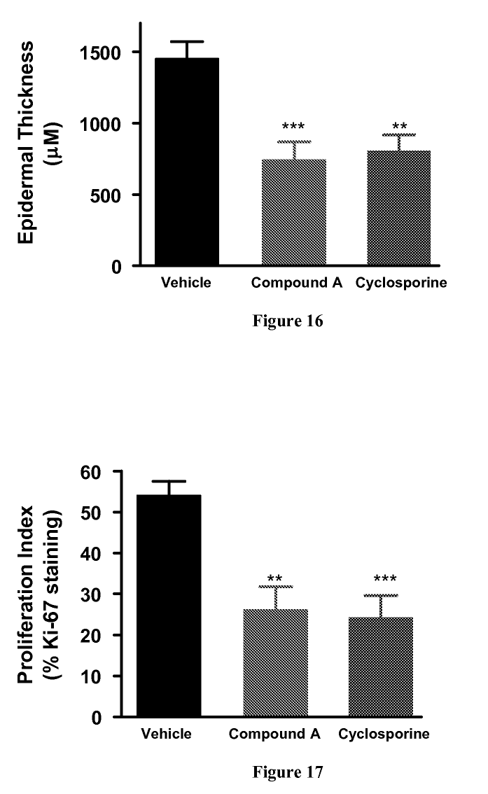

Further, epidermal thickness was used as an indicator of psoriasis features. Vehicle treated beige-SCID mice with normal human skin grafts and injected psoriatic NK cells had a mean epidermal thickness of 1450 microns (FIG. 16 and Table 7). Bars values represent the mean±SEM of 7 beige-SCID mice. However, normal skin grafted/psoriatic NK cells injected beige-SCID mice treated with Compound A, or cyclosporine exhibited an approximate 50% decrease in epidermal thickness compare to vehicle treated animals with mean values of 736, and 804 microns, respectively (FIG. 16 and Table 7). The decrease in epidermal thickness observed in the drug treated mice was significant (P<0.0002; 1-way ANOVA). Specifically, the drug induced decreases in epidermal thickness was significant when compared to the vehicle treated animals (P<0.001 for Compound A; P<0.01 for cyclosporine, Bonferroni Multiple Comparison post-hoc test) (FIG. 16). Compound A performed equivalently to cyclosporine thus no differences in activity were observed (cyclosporine vs Compound A: P>0.05).

Proliferation index, expressed as a percentage of Ki-67 positive keratinocytes using immunohistochemical methods was also used as an additional psoriasis indicator. Bars values represent the mean±SEM of 7 beige-SCID mice. Vehicle treated normal skin grafted/psoriatic NK cells injected beige-SCID mice had 54.1% of keratinocytes expressing the Ki-67 protein, indicative of active cell proliferation (FIG. 17 and Table 7). In contrast, the normal skin grafted/psoriatic NK cells injected beige-SCID mice treated with Compound A, or cyclosporine exhibited decreases (>50%) in the keratinocyte proliferation index to 26.2, and 24.2%, respectively (FIG. 17 and Table 7). These data demonstrated significant reductions in psoriatic NK cells driven proliferating keratinocytes mediated by Compound A and cyclosporine (P<0.0005; 1-way ANOVA). Specifically, the keratinocyte proliferation index was significantly decreased in the Compound A and cyclosporine treated groups compared to vehicle treated animals (p<0.01 and 0.001, respectively, Bonferroni Multiple Comparison post-hoc test) (FIG. 7). However, the differences between Compound A and cyclosporine groups were not significant, indicating that the overall activity of these agents was similar (cyclosporine vs Compound A: p>0.05).

TABLE 7
Summary of Histological Evaluation, Epidermal Thickness and
Keratinocyte Proliferation of each Human Skin Graft

| | | | Epidermal | |
|---|---|---|---|---|
| | | Histological | Thickness | Proliferation |
| Patient | Treatment | Evaluation | (μm) | Index (%) |
| 1 | Vehicle | Psoriasiform | 1314 | 43.5 |
| 2 | Vehicle | Psoriasiform | 1981 | 57.2 |
| 3 | Vehicle | Psoriasiform | 1572 | 65.1 |
| 4 | Vehicle | Psoriasiform | 1520 | 47.6 |
| 5 | Vehicle | Psoriasiform | 1366 | 49.9 |
| 6 | Vehicle | Psoriasiform | 1493 | 61.5 |
| 7 | Vehicle | Psoriasiform | 905 | |
| (Control) Mean | | (0/7) | 1450 | 54.1 |
| 1 | Compound A | Partial recovery w/ | 687 | 41.9 |
| | | dermal infiltration | | |
| 2 | Compound A | Psoriasiform | 1098 | 47.4 |
| 3 | Compound A | Psoriasiform | 626 | 33.5 |
| 4 | Compound A | Complete recovery | 333 | 12.2 |
| 5 | Compound A | Complete recovery | 457 | 9.2 |
| 6 | Compound A | Complete recovery | 627 | 21.4 |
| 7 | Compound A | Psoriasiform | 1324 | 17.5 |
| (Recovery) Mean | | (4/7) | 736 | 26.2 |
| 1 | Cyclosporine | Partial recovery | 768 | 35.2 |
| 2 | Cyclosporine | Psoriasiform | 1083 | 21.8 |
| 3 | Cyclosporine | Psoriasiform | 1027 | 47.2 |
| 4 | Cyclosporine | Psoriasiform | 885 | 28.8 |
| 5 | Cyclosporine | Complete recovery | 376 | 9.0 |
| 6 | Cyclosporine | Psoriasiform | 1078 | 20.7 |
| 7 | Cyclosporine | Complete recovery | 409 | 6.8 |

| | | | |
|---|---|---|---|
| (Recovery) Mean | (3/7) | 804 | 24.2 |

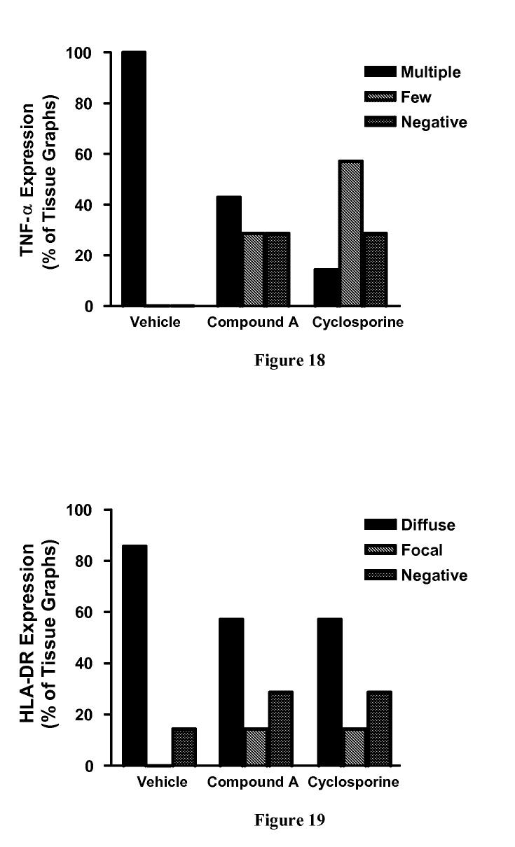

Inflammatory Marker Evaluation of Normal Human Skin Grafts From Psoriatic NK cell Injected Beige-SCID Mice: TNF-α, a pro-inflammatory cytokine is increased in the skin lesions of psoriatic patients. In this study, 7/7 (100%) graphs from vehicle treated mice showed a high level of TNF-α expression in multiple cells (FIG. 18 and Table 8). Down regulation of TNF-α expression was observed in the drug treatment groups. Bars values represent the number of responding graphs divided by the total number of graphs in the treatment group (responding graphs/7 total graphs) and expressed as a percentage. In particular, Compound A treatment group showed 4/7 grafts had either few ( 2/7) or negative ( 2/7) TNF-α expressing cells, demonstrating a partial and complete recovery in 57% of the graphs (FIG. 18 and Table 8). Cyclosporine reduced TNF-α expression by 85.7% ( 6/7 graphs) with partial and complete recoveries in 4/7 (57.1%; few) and 2/7 (28.6%; negative) graphs, respectively (FIG. 18 and Table 8). These data show that both Compound A and cyclosporine have a potential to alleviate psoriasis inflammatory symptoms.

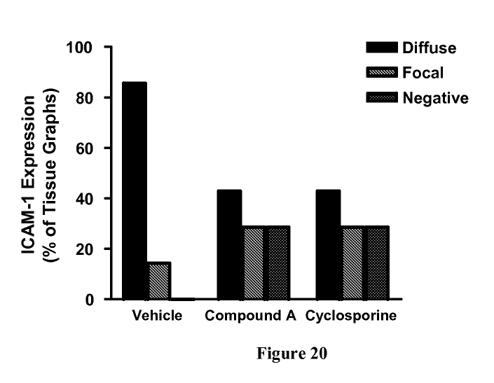

Additional pro-inflammatory markers that are elevated in psoriatic patients are HLA-DR and ICAM-1. Normal skin grafted onto beige-SCID mice injected with psoriatic patient NK cells and treated with vehicle had diffuse HLA-DR and ICAM-1 expression patterns throughout the epidermis in 6/7 (85.7%) graphs (Note: one graph from the vehicle treatment group displayed negative HLA-DR and focal ICAM-1 expression) (FIG. 19, FIG. 20, and Table 8). Bars values represent the number of responding graphs divided by the total number of graphs in the treatment group (responding graphs/7 total graphs) and expressed as a percentage. Compound A and cyclosporine treatment reduced HLA-DR and ICAM-1 expression. In particular, HLA-DR was reduced to focal or negative (0%) expression in 43% of graphs from the Compound A and cyclosporine treated groups. The resulting 43% recovery in the Compound A and cyclosporine treated groups was split with 1/7 (14.3%) and 2/7 (28.6%) displaying focal and negative expression, respectively (FIG. 19 and Table 8). Correspondingly, ICAM-1 expression was reduced to focal or negative expression in 4/7 (57%) graphs ( 2/7 focal and 2/7 negative) from mice treated with either Compound A or cyclosporine. (FIG. 20 and Table 8).

TABLE 8
Summary of Immunohistochemical Staining of Inflammation Markers.
Numbers shown are the total number of grafts expressing each marker

| Inflammation | | | |
|---|---|---|---|
| Marker | Vehicle | Compound A | Cyclosporine |
| TNF-α | 7 Multiple | 3 Multiple | 1 Multiple |
| | 0 Few | 2 Few | 4 Few |
| | 0 Negative | 2 Negative | 2 Negative |
| HLA-DR | 6 Diffuse | 4 Diffuse | 4 Diffuse |
| | 0 Focal | 1 Focal | 1 Focal |
| | 1 Negative | 2 Negative | 2 Negative |
| ICAM-1 | 6 Diffuse | 3 Diffuse | 3 Diffuse |
| | 1 Focal | 2 Focal | 2 Focal |
| | 0 Negative | 2 Negative | 2 Negative |

Diffuse = diffuse pattern throughout the epidermis;
Few = few TNF-α positive cells;
Focal = focal pattern of expression;
Multiple = multiple TNF-α positive cells;
Negative = negative expression (0%).

In summary, the epidermal thickness and proliferation index data, yielded statistically significant results for Compound A suggesting favorable outcomes as a psoriasis treatment. The immunohistochemical staining data partially illustrated the positive mechanistic effects of Compound A in psoriasis. Together, these data suggests that the human skin xenotransplant/SCID mouse model may serve as a tool for investigating potential agents directed against the pathophysiologic mechanisms of psoriasis. The effects of Compound A in the histological and immunohistochemical evaluations suggest that Compound A is efficacious as a psoriasis treatment.

All of the references cited herein are incorporated by reference in their entirety. While the invention has been described with respect to the particular embodiments, it will be apparent to those skilled in the art that various changes and modifications can be made without departing from the spirit and scope as recited by the appended claims.

The embodiments described above are intended to be merely exemplary and those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. A method of treating psoriasis or psoriatic arthritis, which comprises administering to a patient having psoriasis or psoriatic arthritis a therapeutically effective amount of cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable salt or solvate thereof, substantially free of its (R) enantiomer.

2. The method of claim 1, wherein the patient is administered with cyclopropyl-N-{2-[(1S)-1-(3-ethoxy-4-methoxyphenyl)-2-(methylsulfonyl)ethyl]-3-oxoisoindoline-4-yl}carboxamide having the formula:

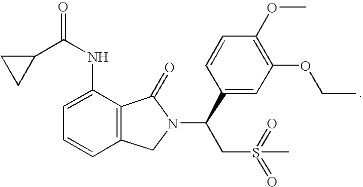

3. The method of claim 1, wherein the cyclopropyl-N-{2-[(1S)-1-(3-ethoxy-4-methoxyphenyl)-2-(methylsulfonyl)ethyl]-3-oxoisoindoline-4-yl}carboxamide is administered as a pharmaceutically acceptable salt.

4. The method of claim 1, wherein the cyclopropyl-N-{2-[(1S)-1-(3-ethoxy-4-methoxyphenyl)-2-(methylsulfonyl)ethyl]-3-oxoisoindoline-4-yl}carboxamide is administered as a pharmaceutically acceptable solvate.

5. The method of claim 4, wherein the cyclopropyl-N-{2-[(1S)-1-(3-ethoxy-4-methoxyphenyl)-2-(methylsulfonyl)ethyl]-3-oxoisoindoline-4-yl}carboxamide is administered as a pharmaceutically acceptable hydrate.

6. The method of claim 1, further comprising administering to the patient a therapeutically effective amount of a second active agent.

7. The method of claim 6, wherein the second active agent is an anti-inflammatory agent, an immnunosuppressant, mycophenolate mofetil, a biologic agent, or a Cox-2 inhibitor.

8. The method of claim 7, wherein the second active agent is etanercept.

9. The method of claim 1, wherein the cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable salt or solvate thereof is administered orally.

10. The method of claim 9, wherein the compound is administered in a dosage form of a tablet or a capsule.

11. The method of claim 1, wherein the cyclopropyl-N-{2-[(1S)-1-(3-ethoxy-4-methoxyphenyl)-2-(methylsulfonyl)ethyl]-3-oxoisoindoline-4-yl}carboxamide, or a pharmaceutically acceptable salt or solvate thereof is administered topically.

12. The method of claim 11, wherein the compound is administered in a dosage form of a lotion or a liquid.

13. The method of claim 1, wherein the therapeutically effective amount is from about 1 mg to about 1,000 mg per day.

14. The method of claim 13, wherein the therapeutically effective amount is from about 5 mg to about 500 mg per day.

15. The method of claim 14, wherein the therapeutically effective amount is from about 10 mg to about 200 mg per day.

16. The method of claim 1, wherein the therapeutically effective amount is about 20 mg per day.

17. The method of claim 16, wherein the compound is administered once or twice per day.

18. The method of claim 1, wherein the therapeutically effective amount is from about 0.01 mg to about 100 mg per kg of a body weight of the patient per day.

19. The method of claim 18, wherein the therapeutically effective amount is about 1 mg, 5 mg or 25 mg per kg of a body weight of the patient per day.

20. A method of treating psoriasis, which comprises administering to a patient having psoriasis a therapeutically effective amount of cyclopropyl-N-{2-[(1S)-1-(3-ethoxy-4-methoxyphenyl)-2-(methylsulfonyl)ethyl]-3-oxoisoindoline-4-yl}carboxamide, substantially free of its (R) enantiomer.
